(19) United States
(12) Patent Application Publication
(10) Pub. No.: US 2026/0355298 A1
(43) Pub. Date: Nov. 12, 2026

(54) GENETICALLY MODIFIED GARDNERELLA SPECIES, COMPOSITIONS, METHODS OF MAKING, AND METHODS OF USE THEREOF

(71) Applicant: Wisconsin Alumni Research Foundation, Madison, WI (US)

(72) Inventors: Joseph Patrick Dillard, Lodi, WI (US); Amy Klimowicz, Madison, WI (US); Erin Michelle Garcia, West Chester, OH (US)

(73) Assignee: Wisconsin Alumni Research Foundation, Madison, WI (US)

(21) Appl. No.: 19/665,421

(22) Filed: May 1, 2026

Related U.S. Application Data
(60) Provisional application No. 63/799,887, filed on May 5, 2025.

(51) Int. Cl. A61K 39/02; A61K 39/00; C12N 1/20; C12N 15/74

(52) U.S. Cl. CPC A61K 39/0208; C12N 1/20; C12N 15/74; A61K 2039/52; A61K 2039/54; A61K 2039/542; A61K 2039/55505; A61K 2039/55583

(57) ABSTRACT

Described herein are genetically modified Gardnerella strains, wherein the genetically modified Gardnerella strains have an insertion, a point mutation, a deletion, or a combination thereof, in a gene encoding a virulence factor, an adhesin, or a biofilm factor. Also described are pharmaceutical compositions including the genetically modified Gardnerella strains and a pharmaceutically acceptable carrier, and their use to treat diseases associated with an imbalance of female reproductive system microbiota such as bacterial vaginosis. Further described are optimized methods for preparing genetically modified Gardnerella strains.

### CROSS-REFERENCE TO RELATED APPLICATIONS

This application claims priority to U.S. Provisional Application 63/799,887 filed on May 5, 2025, which is incorporated herein by reference in its entirety.

### STATEMENT REGARDING FEDERALLY SPONSORED RESEARCH & DEVELOPMENT

This invention was made with government support under AI153534 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Apr. 30, 2026, is named “SEQ_LIST—107668361-P240086US02-updated” and is 59,754 bytes in size. The Sequence Listing does not go beyond the disclosure in the application as filed.

### FIELD OF THE DISCLOSURE

The present disclosure is related to methods of genetically modifying Gardnerella species, the genetically modified Gardnerella strains produced using the methods, compositions comprising the genetically modified Gardnerella strains, and methods of using the genetically modified Gardnerella strains.

### BACKGROUND

Genetic manipulation is a fundamental process in the study of bacterial pathogenesis. The ability to make mutations and complements are important for definitive identification of virulence genes, and the ability to make unmarked mutations may be preferable in these studies. Certain bacterial pathogens were considered genetically intractable for many years, such as Mycobacterium tuberculosis or Chlamydia trachomatis, and studies of their virulence mechanisms necessarily focused on epidemiology, immunology, cell biology in the host, and proteomic or transcriptomic analyses. Those classic cases were eventually resolved by the discovery of effective transduction, natural transformation, and chemical transformation methods, greatly facilitating the identification of virulence factors. Gardnerella species have similarly been considered genetically intractable, and like M. tuberculosis and C. trachomatis, Gardnerella have an unusual cell envelope. Similar to other Actinobacteria, Gardnerella are generally considered Gram-positive, but stain Gram-variable because of their unusual and incompletely characterized envelope.

The Gardnerella genus was originally thought to be made up of a single species, Gardnerella vaginalis. Many studies of Gardnerella diversity divided the isolates by clade, ecotype, or cpn60 type, and recent genomic studies have determined that there are at least thirteen species. Six species have been formally named: G. vaginalis, G. piotii, G. swidsinskii, G. leopoldii, G. greenwoodii, and G. pickettii. Differences in the presence or absence of suspected virulence genes between species have led some researchers to propose that some Gardnerella species are pathogens while others are commensals or that different Gardnerella species play different roles in causing the dysbiosis of bacterial vaginosis (BV). Other studies suggest that Gardnerella species are pathogenic when present with other Gardnerella species, i.e., that a mix of two or more Gardnerella species is needed to initiate the dysbiosis of BV.

BV affects nearly one-third of women of reproductive age, causing problems for sexual and reproductive health. BV in pregnant women greatly increases the risk of preterm birth, and preterm birth is the leading cause of infant mortality. Furthermore, women with BV are more likely to acquire a range of sexually-transmitted infections, including gonorrhea, chlamydia, and HIV, and are more likely to transmit HIV. BV is difficult to cure. Although Gardnerella species are sensitive to antibiotics, only 60-70% of patients are cured following primary treatment, and a significant portion of patients who respond to treatment will relapse within a period of months. Thus, better methods are needed to combat this common and poorly-controlled disease. Identification of crucial virulence factors would allow for the development of vaccines or targeted therapeutics.

### BRIEF SUMMARY

In an aspect, described herein is a genetically modified Gardnerella strain, wherein the genetically modified Gardnerella strain has an insertion, a point mutation, a deletion, or a combination thereof, in a gene encoding a virulence factor, an adhesin, or a biofilm factor. Exemplary virulence factors include a vaginolysin or a sialidase.

In another aspect, included herein is a pharmaceutical composition comprising one or more genetically modified Gardnerella strains as described herein, and a pharmaceutically acceptable excipient.

In a further aspect, a method of treating a woman in need of treatment for a disease associated with an imbalance of female reproductive system microbiota comprises administering the pharmaceutical composition described above to the woman.

In another aspect, a method of preparing a genetically modified Gardnerella strain comprises culturing Gardnerella cells in the presence of D-cycloserine (DCS); transforming the cells with a polynucleotide containing a target modification; plating the cells; and isolating the genetically modified Gardnerella strain comprising the target modification.

### BRIEF DESCRIPTION OF THE DRAWINGS

FIGS. 1 A-C show oligo-mediated recombination in ATCC 14018. (1A) Oligo-mediated recombination in ATCC 14018 with oligo rpsL2mut. Competent cells were prepared without DCS treatment and transformed with 10 μg of rpsL2mut. The No DNA control shows the frequency of spontaneous Str^{R} observed in cells electroporated without DNA. (1B) Competent cells were prepared with or without DCS treatment and transformed with 10 μg of oligo rpsL4mut. The geometric mean and standard deviation of three independent assays is shown. (1C) Competent cells were prepared with DCS treatment and transformed with different rpsL mutagenic oligos. The oligos were 44- or 84-nt in length and contained either a 2- or 4-nt change (2mut or 4mut) from the wild-type sequence. The geometric mean and standard deviation of three independent assays is shown. Significance is shown relative to the respective No DNA control or between oligos as indicated by brackets. *P<0.05, **P<0.01, ***P<0.001.

FIGS. 2A-C show analysis of ATCC 14018 transformed with pAKK134 (‘vly’) to produce insertion-duplication mutants. (2A) Schematic of the vly region of ATCC 14018 and pAKK134 integrated via a crossover in vly. (2B) PCR analysis of nine Erm^{R} isolates from the 14018 x pAKK134 transformation with primers (arrows) to the chromosome (vly-F) and to the vector (pmob-R), producing a 1.1-kb product if pAKK134 is integrated at vly. 1 kb+=NEB 1 kb plus DNA ladder, WT=ATCC 14018, neg=No DNA control. (2C) Hemolysis assay of ATCC 14018 and the insertion-duplication mutant, AKK107. Supernatants from overnight growth were concentrated with 30-kDa cut-off columns, and dilutions were incubated with a 1% suspension of human red blood cells. After removal of unlysed cells, the OD₄₁₅ of the supernatants was measured. The average percent hemolysis is shown for three biological replicates. *, significantly different from WT Sm at P<0.05.

FIGS. 3A and B show the HaeIII-like restriction-modification system in ATCC 14018 presents a barrier to plasmid transformation. (3A) Competent cells of ATCC 14018 were transformed with 2 μg of untreated pAKK134 (‘vly’) or 2 μg of pAKK134 that had been treated with HaeIII methyltransferase. The No DNA control shows the frequency of spontaneous Erm^{R} mutants obtained. The geometric mean and standard deviation of four independent assays is shown. (3B) Transformation of insertion-duplication mutant AKK110 (haeIIIR) with 2 μg of untreated or methylated pAKK149 (‘vly’). The No DNA control shows the frequency of spontaneous Spc^{R} mutants obtained. The geometric mean and standard deviation of three independent assays is shown. Significance is shown relative to the No DNA control or between plasmids as indicated by brackets. *P<0.05, **P<0.01.

FIG. 4 shows transformation of insertion-duplication mutant AKK110 (haeIIIR) with ~2 μg of linearized or circular pAKK111 (Tet^{R}). The No DNA control represents the limit of detection, as no colonies were obtained. The geometric mean and standard deviation of three independent assays is shown. Significance is shown relative to the No DNA control or between plasmids as indicated by brackets. **P<0.01, ***P<0.001.

FIG. 5 shows the length of homologous DNA correlates with the frequency of plasmid integration. ATCC 14018 was transformed with a series of plasmids that contain ermC for selection and an internal fragment of lacZ, ranging in size from 0.2-1.8 kb, for homologous recombination with the chromosome. The control represents the frequency of spontaneous Erm^{R} isolates in the sample that did not contain DNA. The geometric mean and standard deviation is shown for four independent assays. Significance is shown relative to the No DNA control or between plasmids as indicated by brackets. *P<0.05, **, P<0.01, ***, P<0.001.

FIGS. 6A-C show construction and characterization of a vly point mutant in ATCC 14018 using a positive/negative selection plasmid. (6A) Schematic depicting construction of a vly point mutant. Plasmid pAKK165 contains ermC for positive selection (plasmid integration) and pheSₘᵤₜ₂ for counter selection (plasmid excision). The 0.7-kb fragment of vly contains a Glu155* mutation, indicated by the “X.” (6B) DNA sequencing confirmed that the single nucleotide change that results in a premature stop (GAA to TAA; E155*) in vaginolysin was present in mutant AKK124 (GATTTGCCGGGGATTCCACGGCGGCTAAAGTGCTGTAACTGTTCAGCGCCC; SEQ ID NO: 35 is the DNA sequence, DLPGFHGGSAVTVQRP; SEQ ID NO: 36 is the corresponding protein sequence). The nt change is highlighted in yellow. (6C) LDH levels released from endocervical (left panel) and ectocervical (right panel) tissues that were inoculated with media alone (NT), ATCC 14018 (WT) or AKK124 (VLY). LDH was quantified after 12 and 24 hr. The mean±standard deviation of four (ectocervix) or three (endocervix) independent assays is shown. Significantly different at *P<0.05, **P<0.01, ***, P<0.001.

FIGS. 7A-D shows markerless deletion of vly in ATCC 14018 using a positive/negative selection strategy. (7A) Schematic depicting the vly region in ATCC 14018 and plasmid pAKK176 used in the two-step gene replacement. pAKK176 contains fragments flanking vly designated “up” and “down” cloned into pAKK174. In the first step, transformants that have integrated the plasmid are selected for by plating on medium with Erm. In this example, integration occurred via a single crossover in the “up” region of the chromosome. In the second step, growth of the integrant without Erm, followed by plating on media containing 4-chloro-DL-phenylalanine (4CP), selected for isolates that had undergone a second recombination event and lost the plasmid. In this example, the second crossover occurred in the “down” region, resulting in deletion of vly. (7B) Isolates that were resistant to 4CP and sensitive to Erm were patched to HBT agar to screen for recombinants that exhibited reduced hemolysis. Patches 2-5 appeared hemolytic, while patches 1 and 6-10 appeared to have diminished activity. The patch labeled “1” was analyzed by PCR. (7C) and (7D) PCR analyses confirming the vly deletion. (7C) PCR with primers that are internal to vly (arrows in A; vly_mid-F/vly_screen-R). (7D) PCR with primers that anneal outside of the “up” and “down” flanking regions (green arrows in A; vly_up_screen-F/Gv_abc-R). Lanes: 1 kb+=DNA ladder; WT=14018; intg=pKK174 integrant; mut=vly deletion mutant AKK132; neg=No DNA control. Note that the PCR extension time in D was not sufficient to allow amplification of the 7.6-kb product from the integrant.

FIG. 8 shows complementation construct for Gardnerella. Regions of homology with the G. vaginalis ATCC 14018 chromosome are shown in gray. pAKK186 contains the rpsB promoter from ATCC 14018 for constitutive expression of a gene cloned into the multiple cloning site (MCS). Plasmid integrants will be Erm^{R} Spc^{R}, while double recombinants will be Erm^{R} Spc^{S}.

FIGS. 9A-D show complementation of vly mutant. (9A) Schematic depicting the complementation region in AKK132, vly complementation construct pAKK187, and the double recombinant. pAKK187 contains vly+ and ermC cloned in between ‘pknB and ‘srtE. A promoter from the ATCC 14018 rpsB gene precedes vly for constitutive expression. Additionally, there is a gene for spectinomycin resistance on the vector. In the first step, transformants of AKK132 (Δvly) containing the plasmid were selected for by plating on media with erythromycin. Erm^{R} isolates were patched to plates containing spectinomycin to identify transformants that had either integrated the plasmid (Erm^{R} Spc^{R}) or had undergone a double crossover (Erm^{R} Spc^{S}), as depicted in the schematic. (9B) PCR analysis confirming the presence of vly at the complementation site in a double recombinant. Primer pairs (shown as arrows in A) that anneal outside of the ‘pknB or ‘srtE fragment and within vly were used to amplify regions~2.4 kb in size. Lanes: 1 kb+=DNA ladder; Δvly=mutant; comp=complemented mutant; neg=negative control. The amplicon from the complemented mutant (9B left) was sequenced, confirming the presence of vly. (9C) Hemolysis phenotypes of wildtype (ATCC 14018), Δvly mutant (AKK132) and complemented mutant (AKK136) on HBT agar. The top panel shows the plate illuminated from ambient light. The bottom panel shows the plate illuminated by a light box from below. (9D) Hemolysis assay of concentrated supernatants from cultures of small colony variants grown overnight in BHI-YDS medium. The mean±standard deviation of three independent assays is shown.

FIGS. 10A-C shows sialidase activity of 3336 and the nanH3 deletion mutant. (10A) Bacterial lysates were mixed with the chromogenic substrate X-Neu5Ac and incubated overnight at 37 °C. Absorbance at OD₆₁₅ was measured. The mean±standard deviation of three independent assays is shown. *, significantly different from WT at P<0.05. (10B) Samples are shown from a representative assay after overnight incubation with X-Neu5Ac. The blank contained sodium phosphate buffer with X-Neu5Ac. (10C) Wells of a microtiter plate were pre-coated with cervical mucus, followed by treatment with 3336 or the nanH3 mutant. Lectin MAL II was used to quantify terminal sialic acids on mucus glycans. Controls included wells that did not receive MAL II (neg) and wells that received MAL II but no bacteria (pos). In addition, bacteria were applied to wells that had not been pre-coated with cervical mucus (untreated). Data were normalized to the positive control. The mean standard deviation of three independent assays is shown. Statistical significance was assessed by the Welch's t-test for comparisons to the positive control, and by the Student's t-test for comparisons between bacterial samples. Significance is shown relative to the positive control or between treatments as indicated by brackets. *P<0.05, **, P<0.01, ***, P<0.001.

The above-described and other features will be appreciated and understood by those skilled in the art from the following detailed description, drawings, and appended claims.

### DETAILED DESCRIPTION

Described herein are genetically modified Gardnerella species as well as a set of tools and methods for genetic manipulation of Gardnerella species. The methods allow for targeted mutagenesis and use selectable markers. The constructs work for strains from two different Gardnerella species showing that they are expected to be generalizable across Gardnerella species. Making plasmid insertions is straightforward using the methods described herein, and maintaining antibiotic selection can allow for such insertion mutations to be stably maintained. Using the positive/negative selection plasmid to deliver point mutations or deletions to the chromosome was achieved. Such mutants are stable and can be designed to have minimal polar effects on nearby genes.

The genetic methods described here will allow researchers to identify virulence factors in the various Gardnerella species and dissect the roles of these factors in development of the dysbiosis and the associated pathology.

Further, the genetically modified Gardnerella stains described herein may be used in pharmaceutical compositions and methods, such as those useful for the treatment and/or prevention of BV.

In an aspect, described herein is a genetically modified Gardnerella strain, wherein the modified Gardnerella strain has an insertion, a point mutation, a deletion, or a combination thereof, in a gene encoding a virulence factor, an adhesin, or a biofilm factor.

Several factors are predicted to act in pathogenesis of Gardnerella species. Gardnerella are proposed to cause the dysbiosis of BV by adhering to epithelial cells, building a biofilm that multiple BV-associated bacteria grow on, lysing human cells, degrading host molecules, cross-feeding other BV-associated bacteria, degrading IgA, and reducing the inflammatory response. Putative adhesins and biofilm factors include Tad pili, sortase-dependent pili, and proteins predicted to bind extracellular matrix and serum molecules including fibronectin, albumin, and collagen. Most Gardnerella produce the toxin vaginolysin which lyses red blood cells, epithelial cells, and neutrophils. Such lysis would release numerous host molecules that the bacteria could use for nutrition and for supplying nutrients to other BV-associated bacteria. Glycogen degradation and protein degradation are described as supplying Gardnerella and the other bacteria in the biofilm with nutrients. Sialidase is present in some Gardnerella species and is proposed to act in the degradation of mucins. Gardnerella sialidase has been shown to act on IgA, potentially leading to IgA destruction. Sialidase activity correlates with BV and is used as a diagnostic factor. Other glycosylases such as fucosidase and beta-galactosidase are likely also involved in mucus degradation.

Exemplary virulence factors include a vaginolysin, a sialidase, a phospholipase C, a prolidase, a glycosulfatase, Tad pili, sortase-dependent pili, fibrontectin-binding proteins, collagen-binding proteins, albumin-binding proteins, beta-galactosidase, fucosidase, and combinations thereof.

Vaginolysin is a pore-forming toxin that lyses human red blood cells, epithelial cells, and neutrophils. Vaginolysin binds human CD59 which may explain the human-specific nature of Gardnerella infection.

Sialidases are enzymes that degrade protective mucins in vaginal mucus, promoting bacterial adhesion and biofilm formation.

Some strains of Gardnerella, such as G. vaginalis produce phospholipase C, an enzyme that hydrolyzes phospholipids and can contribute to the pathogenesis of BV.

Prolidase is an enzyme that breaks down proline-rich peptides.

Glycosulfatases remove sulfate groups from mucin.

Exemplary Gardnerella species include G. vaginalis, G. picketti, G. piotii, G. leopoldii, G. swidsinskii, G. greenwoodii, and the like. G. vaginalis, G. piotii, G. picketti, and G. swidsinskii have particularly been linked to BV.

In an aspect, the genetically modified Gardnerella strain is a G. vaginalis strain comprising an insertion of an antibiotic resistance gene, a nonsense mutation at E155 (GAA to TAA), or a deletion of all or a portion of the vaginolysin gene coding region.

In another aspect, the genetically modified Gardnerella strain is a G. pickettii strain comprising a deletion of all or a portion of a nanH3 sialidase gene.

Additional genetically modified Gardnerella strains include G. vaginalis (e.g., strain 14018) with a haeIIIR deletion, G. vaginalis (e.g., strain 14018) with a FIVAR repeat protein (E3D8E1) deletion, G. vaginalis (e.g., strain 14018) with a pilus pil2 (E2D727) deletion, or G. pickettii (e.g., strain 3336) with a sialidase (nanH3) deletion.

In one embodiment, the genetically modified Gardnerella strain is lyophilized, or freeze-dried in a manner that preserves bacterial viability. Methods of preserving viable bacteria by lyophilization can promote long-term preservation of the microorganism. One skilled in the art will be able to lyophilize bacteria using standard techniques. Briefly, microbes are cultured and suspended in lyophilizing buffer or medium. The microbes are rapidly frozen and then subjected to a primary and secondary drying phase to remove all readily available water and residual water, respectively.

Also included are pharmaceutical compositions comprising the genetically modified Gardnerella strains and a pharmaceutically acceptable excipient. Pharmaceutical compositions can be prepared in a dosage form such as, for example, tablets, capsules, nasal sprays, powders, granules, ointments, solutions, suppositories, injections, inhalants, gels, microspheres, and aerosols. Exemplary dosage forms include oral dosage forms such as a tablet or a capsule or an oral liquid dosage form.

Pharmaceutically acceptable excipients include binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof.

In an aspect, a pharmaceutical composition is suitable for administration to the vagina, that is, a vaginal dosage form. Examples of vaginal delivery methods include vaginal tablets, capsules, suppositories, creams, and douches. In one embodiment, the composition will be preserved by drying on film, for example preservation by vaporization (PBV).

The pharmaceutical composition can be formulated for controlled- or extended-release. Advantages of controlled-release formulations can include extended activity of the drug or active substance; reduced dosage frequency; increased patient compliance; administration of less total drug or active substance; reduction in local or systemic side effects; reduction in blood level fluctuations; improved efficacy of treatment; reduction of potentiation or loss of drug activity; and/or improvement in speed of control of diseases or conditions.

Controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this constant level, active ingredient should be released from the dosage form at a rate that will replace the amount of active ingredient being metabolized or otherwise lost from the body. Controlled-release of an active ingredient can be stimulated by various conditions including, but not limited to, pH, ionic strength, osmotic pressure, temperature, enzymes, water, and other physiological conditions or compounds.

A variety of known extended-release dosage forms, formulations, and devices can be adapted for use with viable bacterial compositions described herein. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In another embodiment, compositions can include encapsulation technology, including, but not limited to a gel composition.

Another exemplary dosage form is a suppository. The term “suppository” refers to dosage forms or formulations that are inserted into body cavities. Suppositories are solid bodies of various sizes and shape, which dissolve, disintegrate or melt upon administration. Vaginal suppositories are designed for insertion into a vaginal cavity or urogenital tract, with or without an applicator. For example, vaginal suppositories can be inserted with an applicator high into the vaginal tract or with or without an applicator into the vaginal cavity. Vaginal suppositories are usually globular, oviform, or cone-shaped, but can be of any shape or size suitable for insertion into the vaginal cavity. A tablet, a ring or a capsule for insertion into vaginal cavity or urogenital tract is included in the scope of the term “suppository.”

Optionally, a vaginal delivery system may include a means for administering the composition to the vaginal cavity. It may be comprised of a device or dosage form for vaginal delivery of the compositions comprising a pharmaceutical composition as described herein. For example, a suitable device may comprise an open end (e.g., a tip) for insertion into the vaginal cavity, and a dispensing end (e.g., a plunger or piston) to expel the composition through the open end. The device could then be used directly for administration (if pre-filled) or filled with the composition immediately (or shortly prior to) administration by a user. Alternatively, the device is filled with the composition and stored under refrigeration for a few days up to about a week (or alternatively frozen if it is stored for longer periods) prior to administration. In some cases, e.g., where the device is an applicator or dispenser, after release of the composition in the vaginal cavity, the device is removed from the vaginal cavity. Exemplar devices include applicators and dispensers, e.g., those commonly used in medicine and science. The applicators and dispensers may either be operated manually or comprise electromechanical aides.

Additional vaginal delivery systems may comprise an absorbent product comprising the composition comprising a substantially complete vaginal microbiota preparation such as, e.g., a feminine hygiene diaper, panty liners, sanitary napkin, tampon, panty guard or an incontinence guard.

In an aspect, the pharmaceutical composition further comprises one or more additional bacteria, such as Bifdobacteria, Lactobacillus species bacteria. Exemplary additional bacteria include Lactobacillus acidophilus, Lactobacillus rhamnosus, Lactobacillus reutri, Lactobacillus fermentum, Lactobacillus paracasei, Lactobacillus brevis, Lactobacillus salivarius, Lactobacillus plantarum, Lactobacillus crispatus, Lactobacillus iners, Lactobacillus jensenii, Lactobacillus gasseri, Lactobacillus casei, and combinations thereof.

OPositive Health, for example, markets a vaginal probiotic blend comprising Lactobacillus acidophilus, Lactobacillus rhamnosus, Lactobacillus reutri, Lactobacillus fermentum. The Seed VS-01™ vaginal symbiotic includes Lactobacillus crispatus. Advantageously, the genetically modified Gardnerella strains described herein could be added to these vaginal bacteria blends.

In some embodiments, the pharmaceutical compositions further comprise one or more agents that promote bacterial growth. Non-limiting examples of agents that promote bacterial growth include boric acid, a prebiotic, and low pH buffering agents as sodium bicarbonate and agents that can act as both acidifying and prebiotics, such as ascorbic acid (vitamin C).

Prebiotics promote the growth, survival, and activity of beneficial microorganisms, for example Bifdobacteria and Lactobacillus. Prebiotics have been shown to alter the compositions of microorganisms (microflora) in the gut microbiome, and are contemplated for assisting in the maintenance of a healthy vaginal flora, alone or in combination with Lactobacillus cocktail compositions, for example. In addition, prebiotics have been shown to increase calcium and magnesium absorption in the gut, increase bone density, enhance the immune system, reduce blood triglyceride levels, and control hormone levels. Prebiotics include any of a number of compositions that are generally not directly digestible by humans, but that are readily digestible by and promote the growth or establishment of probiotic microbes. Topical vaginal application of the prebiotic sucrose is an effective therapeutic in patients with symptomatic BV. Non-limiting examples of other prebiotics include but are not limited to inulin, fructooligosaccharides, galactooligosaccharides, hemicelluloses (e.g., arabinoxylan, xylan, xyloglucan, and glucomannan), chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber), xylooligosaccharides, and combinations thereof.

The vaginal pH ranges from 3.8 to 4.5 in a healthy subject. This acidic pH promotes the growth of beneficial bacterial (for example Lactobacillus) and prevents the overgrowth of vaginal pathogens that cause odor, irritation, and infection. Buffering agents are weak acids or bases that maintain the acidity at a chosen level and prevent a rapid change in acidity. Non-limiting examples of low pH buffering agents are lactic acid and sodium bicarbonate.

In an aspect, the pharmaceutical composition is a vaccine composition. The non-toxigenic genetically modified Gardnerella strain could serve as a vaccine against pathogenic Gardnerella. Without being held to theory, it is believed that the genetically modified Gardnerella strains described herein are non-pathogenic to the individual to whom they are administered and can be used to stimulate an immune response to endogenous Gardnerella strains in the individual. In an aspect, the use of the genetically modified Gardnerella strain reduces and/or eradicates the endogenous Gardnerella strains in the individual.

A vaccine composition could be delivered by subcutaneous (sc) delivery, intramuscular (im) delivery, intradermal delivery, transdermal delivery, mucosal delivery, intravaginal delivery, intrarectal delivery, intraperitoneal delivery, inhalation delivery, and/or aerosol delivery.

A method of treating a woman in need of treatment for a disease associated with an imbalance of female reproductive system microbiota comprises administering the genetically modified Gardnerella strain described above to the woman.

Exemplary diseases associated with an imbalance of female reproductive system microbiota include bacterial vaginosis, pelvic inflammatory disease, sexually transmitted infection, postpartum endometritis, acute endometritis, chronic endometritis, postpartum sepsis, likelihood of preterm birth, likelihood of preterm premature rupture of membranes, likelihood of miscarriage, miscarriage, recurrent miscarriage, chorioamnionitis, intra-amniotic infection, infertility, sterility, likelihood of ineffectiveness of fertility treatment, implantation failure, ectopic pregnancy, placenta previa, placental abruption, intrauterine growth restriction, pre-term delivery, preeclampsia, premature membrane rupture cervical intraepithelial neoplasia, cervical cancer, other cancers of the female genitourinary system, decreasing ammonia in the vaginal environment, reducing inflammation, preventing overgrowth of at least one of Gardnerella vaginalis and Prevotella spp. in the vagina, and combinations thereof.

In an aspect, the woman has BV or is at risk of developing BV. BV in pregnant women greatly increases the risk of preterm birth, and preterm birth is the leading cause of infant mortality. Furthermore, women with BV are more likely to acquire a range of sexually-transmitted infections, including gonorrhea, chlamydia, and HIV, and are more likely to transmit HIV.

In another aspect, a method of preparing a genetically modified Gardnerella strain comprises culturing Gardnerella cells in the presence of D-cycloserine (DCS); transforming the cells with a polynucleotide comprising a target modification; plating the cells; and isolating the genetically modified Gardnerella strain comprising the target modification.

In an aspect, culturing Gardnerella cells in the presence of D-cycloserine (DCS) increases transformation efficiency, possibly by reducing the extent of crosslinking between peptidoglycan strands and thereby weakens the barrier function of the cell envelope.

An aspect, cells are transformed with a linear polynucleotide comprising an antibiotic resistance marker in it (such as ermC) or ermC together with pheS (mut2alt), or a linear construct containing a mutation sequence to target the gene of interest.

In an aspect, the cells are transformed with a circular polynucleotide comprising a target modification, such as an insertion, a point mutation, a deletion, or a combination thereof. As shown herein, circular polynucleotides can increase transformation efficiency compared to linear polynucleotides. In addition to the target modification, the circular polynucleotide can include DNA sequences to guide insertion of the target modification into the Gardnerella strain genome.

In yet another aspect, it was found that cleavage by a HaeIII-like type II restriction-modification (RM) system that cleaves at unmethylated GGCC sequences might be preventing plasmid transformation. Thus, in an aspect, the circular plasmid is methylated, e.g., with HaeIII methyltransferase. In another aspect, the Gardnerella cells are HaeIII-like endonuclease gene-deficient.

In an aspect, the circular polynucleotide containing the target modification comprises an ermC gene for selection of polynucleotide integration and a mutated pheS gene (T251S A294G) for selection of polynucleotide loss, optionally wherein the mutated pheS gene is codon optimized for Bifidobacterium longum.

In another aspect, the circular polynucleotide is an E. coli plasmid. In an aspect, the E. coli plasmid comprises a ermC gene, a polylinker, an E. coli origin of replication, and optionally two copies of the neisserial DNA Uptake Sequence (DUS).

The ermC gene encodes a methyltransferase that catalyzes the methylation of A2058 of the 23S ribosomal RNA in two steps. Expression of ErmC is inducible by erythromycin.

An E. coli origin of replication can be a 245-bp sequence that is the starting point for DNA replication in E. coli.

In Neisseria bacteria, DNA Uptake Sequences (DUSs) are short DNA sequences that enhance the efficiency of natural transformation, the process by which bacteria take up foreign DNA from the environment. DUSs act as a signal for the bacterial machinery responsible for taking up DNA. DUSs are typically 10-12 base pairs long, with variations in sequence and structure.

In an aspect, the E. coli plasmid is pIDN1, pIDN2, pIDN3, or pIDN4.

The invention is further illustrated by the following non-limiting examples.

### EXAMPLES

### Methods

Bacterial strains, oligos, plasmids, primers, and growth conditions: Bacterial strains and plasmids used in this study are presented in Table 1. Gardnerella vaginalis ATCC 14018 was used to develop a method for electrotransformation starting with the methods for transformation with oligos described in the art. Gardnerella species exhibit phase variation in colony size; due to the faster growth rate of the large colony variant, the large morphotype was used for these studies. G. vaginalis was initially streaked from frozen stocks onto human blood tween (HBT) plates (Thermo Scientific™) and grown overnight at 37 °C with 5% CO₂. To prepare electrocompetent cells, ATCC 14018 was grown in BHI-YDS media, which consisted of brain heart infusion (BHI) supplemented with 0.5% yeast extract, 0.2% glucose, and 0.1% starch. BHI-YDS agar plates were supplemented with 10% FBS and when necessary, antibiotics at the following concentrations: 50 or 100 μg/ml streptomycin (Str); 0.03 μg/ml erythromycin (Erm); 16 μg/ml spectinomycin (Spc); 8 μg/ml tetracycline (Tet). BHI agar supplemented with 10% FBS and 5% Fildes Enrichment, designated BHIFF medium, was used as nonselective medium for G. vaginalis. E. coli cultures were grown in LB media containing the appropriate antibiotic at the following concentrations: 500 μg/ml Erm; 100 μg/ml Spc. Primers, oligos, and gblocks (Table 2.) were purchased from Integrated DNA Technologies.

TABLE 1
Bacterial strains and plasmids used in this study.

| | | Source/ |
|---|---|---|
| Strain or plasmid | Characteristics | reference |
| Bacterial strains | | |
| Gardnerella spp. | | |
| ATCC 14018 | G. vaginalis LacZ⁺, hemolytic, sialidase⁻ | ATCC |
| 3336 | G. pickettii LacZ⁻, hemolytic, sialidase⁺, Tet^{R} | M. Roberts |
| AKK107 | 14018 × pAKK134 (‘vly’) ID* mutant, Erm^{R} | This work |
| AKK110 | 14018 × pAKK139 (‘haeIII’) ID mutant, Erm^{R} | This work |
| AKK112 | 14018 × pAKK144 (0.2 kb ‘lacZ’) ID mutant, Erm^{R} | This work |
| AKK113 | 14018 × pAKK145 (0.5 kb ‘lacZ’) ID mutant, Erm^{R} | This work |
| AKK114 | 14018 × pAKK146 (1 kb ‘lacZ’) ID mutant, Erm^{R} | This work |
| AKK115 | 14018 × pAKK148 (1.8 kb ‘lacZ’) ID mutant, Erm^{R} | This work |
| AKK123 | 14018 × pAKK165 (vlymut pheSₘᵤₜ₂) integrant, Erm^{R} | This work |
| | 4CP-sensitive | |
| AKK124 | 4CP-resistant Erm^{S} AKK123, vly point mutant | This work |
| AKK131 | 14018 × pAKK176 (Δvly pheSₘᵤₜ₂_{_{—}}ₐₗₜ) integrant | This work |
| AKK132 | 4CP-resistant AKK131, Δvly mutant | This work |
| AKK136 | AKK132 × pAKK187 (Δvly/vly+) complemented | This work |
| | mutant | |
| AKK140 | 3336 × pAKK191 (ΔnanH3 pheSₘᵤₜ₂_{_{—}}ₐₗₜ) integrant, | This work |
| | Erm^{R} | |
| AKK141 | 4CP-resistant AKK141, ΔnanH3 mutant | This work |
| E. coli | | |
| TAM1 | Used for cloning | RapidTrans ™ |
| TOP10 | Used for cloning | Invitrogen ™ |
| Plasmids | | |
| pIDN1 | E. coli cloning vector, Erm^{R} | |
| pSPUC | E. coli cloning vector, source of spectinomycin | |
| | resistance gene, Spc^{R} | |
| pAKK134 | 0.5 kb ‘vly’ in pIDN1, Erm^{R} | This work |
| pAKK139 | 0.45 kb ’haeIII’ in pIDN1, Erm^{R} | This work |
| pAKK144 | 0.2-kb ‘lacZ’ in pIDN1, Erm^{R} | This work |
| pAKK145 | 0.5-kb‘lacZ’ in pIDN1, Erm^{R} | This work |
| pAKK146 | 1-kb lacZ’ in pIDN1, Erm^{R} | This work |
| pAKK148 | 1.8-kb ‘lacZ’ in pIDN1, Erm^{R} | This work |
| pAKK163 | counter selectable construct 1; pheSₘᵤₜ₂ gblock in | This work |
| | pIDN1, Erm^{R} | |
| pAKK165 | ’vlymut’ (point mutation) gblock cloned into | This work |
| | pAKK163, Erm^{R} | |
| pAKK174 | counter selectable construct 2; pheSmut2_alt gblock | This work |
| | cloned into pIDN1, Erm^{R} | |
| pAKK176 | Δvly gblock cloned into pAKK174, Erm^{R} | This work |
| pAKK182 | Spec cassette cloned into XhoI/NsiI sites of pIDN1 | This work |
| | (replacing ermC), Spc^{R} | |
| pAKK185 | ‘pknB_‘srtE gblock cloned into pAKK182, Spc^{R} | This work |
| pAKK186 | complementation plasmid for G. vaginalis; ermC | This work |
| | cloned into pAKK185, Spc^{R} Erm^{R} | |
| pAKK187 | vly+ cloned into pAKK186, Spc^{R} Erm^{R} | This work |
| pAKK191 | ΔnanH3 from 3336 cloned into pAKK174, Erm^{R} | This work |

*ID = insertion-duplication mutant

TABLE 2
Sequences of primers, oligos and gblocks used in this study*.

| | | SEQ ID |
|---|---|---|
| Primers | Sequence 5′-3′ | NO: |
| vly SacI-F | CTCTGAGCTCCTCAAATTGTGAACTTCAAGC | 1 |
| Gv abc-R | TGAACGCTCAACACTTGC | 2 |
| vly-F | CATATGAAGAGTACAAAGTTCTACC | 3 |
| pmob-R | AACAGCTATGACCATGATTACGCCAAG | 4 |
| vly screen-F | CGTCAAGGATAACGAAGTAGC | 5 |
| vly screen-R | GCTACTTCGTTATCCTTGACG | 6 |
| Gv haeIII SacI | GATAGAGCTCGGATCTAACTGGCAAGAGA | 7 |
| -F | | |
| Gv haeIII ClaI | GCTCATCGATCAAAGGTCTGTCACCATC | 8 |
| -R | | |
| haeIII screen-F | AGGCATTGGACTGCAAACTGG | 9 |
| Gv lacZ SacI- | CTATGAGCTCGCATCATGGCTAGAGGATC | 10 |
| F | | |
| Gv_lacZ(0.5 kb) | TGATCTCGAGCACATCGGAAACCATGTCA | 11 |
| XhoI-R | | |
| Gv_lacZ(1 kb) | GTATCTCGAGTCTGGGTTGTTTGACACG | 12 |
| XhoI-R | | |
| Gv lacZ-R3 | TGTAGCGTATTGTCGTCG | 13 |
| spec ClaI-F | AGATATCGATTAACGTGACTGGCAAGAG | 14 |
| spec NsiI-R | GACAATGCATACAGCTATGACCATGATTACG | 15 |
| vly SpeI-F | CTATACTAGTCTGTAACTTCCGCAGTGA | 16 |
| vly mid-F | TTCGATGCAGTACACAAGG | 17 |
| vly_up_screen- | TGTGTGATGCGTGACCTTC | 18 |
| F | | |
| pknB screen2- | GCTGGAGAAGTTGTAGTTCC | 19 |
| F | | |
| srtE screen3-R | GACCAGAACAGGTTGAGG | 20 |
| nanH3_up_SacI | CATAGAGCTCAGCACGCAGTAAATAGACC | 21 |
| -F | | |
| nanH3_up_SpeI | GCATACTAGTCGCTGTTCCAATCATTATATTCC | 22 |
| -R | | |
| 3336nanH3 do | CAGTACTAGTGGAATCAACACAACTATTCCTTATGC | 23 |
| wn SpeI-F | | |
| 3336nanH3 do | TTAACTCGAGAACTAAGATGCCCTCGGT | 24 |
| wn XhoI-R | | |
| Oligos | | |
| rpsL2mut | CGTGTGTACACCACCACTCCTCGGAAGCCAAACTCTG | 25 |
| | CGCTTCG | |
| rpsL4mut | CGTGTGTACACCACCACTCCACGCAAGCCAAACTCTG | 26 |
| | CGCTTCG | |
| rpsL2mut_long | CGTGGCGTGTGCACTCGTGTGTACACCACCACTCCTC | 27 |
| | GGAAGCCAAACTCTGCGCTTCGTAAGGTTGCTCGTGT | |
| | GCGCCTCAGC | |
| rpsL4mut_long | CGTGGCGTGTGCACTCGTGTGTACACCACCACTCCAC | 28 |
| | GCAAGCCAAACTCTGCGCTTCGTAAGGTTGCTCGTGT | |
| | GCGC CTCAGC | |
| gblocks | | |
| 0.2 kb ‘lacZ’ | CTATGAGCTCGCATCATGGCTAGAGGATCAAGATTAT | 29 |
| gblock | TGGAGACTACACGGCATTTTCCGTTCCGTTGAACTGT | |
| | GTGCACATCCTTCCACCCATGTATCAAATCTTCATGTT | |
| | GATGCAGATTATAGCAATGATACAAATACTGGGAAA | |
| | CTCGCATTCAGAGCAAACATTGAAGGAGACAATTTA | |
| | AAAGATATTACACTACACGCTTACATCTCGAGGTAT | |
| pheSmut2 | CTGAATCGATCGTAATGCGTCGTACATACCGCACATT | 30 |
| gblock | AAAATGAACATGTTAGTTTAAATAACGAAGACCAAA | |
| | ACGTATTGTTAAAAATCGCGAAAAATCGCTAGAACTA | |
| | TGCGTTAAGGTTTCACTCAAGGGAGATCCCATATGCT | |
| | TCACAAAAGTACACATAACACGGATAGGAAGGGTGC | |
| | TGTGGCAAGTAGTAAGCCGTTCGACGCGAAGGCAAT | |
| | TACAAAGGTAGTAAAAGAGGGTATAGCTTGTGTTCA | |
| | AGCCGCTAAAACAATGGAAGAGTTGAAAGCGGCAAA | |
| | AACTAAGTATGCAGGAGCTCAGTCTGCAATGACCCTT | |
| | GCCAGCAAGTCTATTGGAAGTCTTCAAGTAGAAGAA | |
| | AAGAAAGAAGCTGGCAAGATAATGTCGGCTTTACGC | |
| | GCTGATTTTGGTCGTGAATTTGCAGCAGCCCAAGAGC | |
| | GTATTAAGGCTATTGAAGAAGCTAATATGCTTCAAAA | |
| | AGAAACAGTCGATATGACCTTGCCAATAAATCGTAA | |
| | GCCGCTTGGCGCTCGCCATCCGATTGCGCGTATTATT | |
| | GAAGACTTTGAAGACTTCTTTGTGTCTATGGGTTGGC | |
| | AGATTTCTGCAGGACCAGAGGTAGAAACAGAGTGGT | |
| | TCGATTTTGACGCGTTGAATTTTGGTCCAGATCATCCT | |
| | GCTCGTCAAATGCAAGATACTTTTTATGTGCAAGGTA | |
| | ATCAGGCAAAGGATGCTGCAGGATTTGTTGGATCTAA | |
| | CATGGTTTTGCGTACGCAAACTTCTTCCGATCAGGTT | |
| | CGTGCGTTAATTGAGCGAGGAGTACCTCTGTATATTG | |
| | CGTCCCCAGGCCGAGTATTCCGCACGGATGAGCTTGA | |
| | TGCAACGCATACTCCAGTCTTCCACCAGTGCGAAGCT | |
| | TTGGCAGTAGATAAACATTTGAGCATGGCTGATTTAA | |
| | AGGGTGTTCTTGATCGCCTTGCTGTTGCAATGTTTGGT | |
| | CCAGATGCAAAAAGCCGTTTGCGCCCAAGCTACTTCC | |
| | CGTTCTCTGAGCCTAGTGCGGAGTTGGATCTTTGGTT | |
| | CCCAGATAAGAAGGGTGGCCCAGGTTGGATTGAATG | |
| | GGGCGGATGCGGAATGGTGAATCCGAATGTTCTTAA | |
| | GTCTGCAGGCTTAGACCCAGATGTTTATACGGGATTT | |
| | GGATTCGGTGTTGGCTTGGAGCGCACTTTGCTGCTTC | |
| | GTCACGATATTAACGATATGCACGATTTGGTTGAAGG | |
| | CGATAAGCGCTTCAGTGAACAGTTTGTGATGGGAGA | |
| | GTAGCTCGAGTATG | |
| vlymut gblock | CTGTACTAGTGAACCAGCTACATCTTGCGCAGCTAAG | 31 |
| | AAAGACTCGTTGAATAATTATTTGTGGGATTTGCAAT | |
| | ACGATAAAACAAACATTCTCGCCCGTCATGGCGAAA | |
| | CCATTGAGAACAAATTCTCCAGCGACAGCTTCAACAA | |
| | GAACGGTGAATTCGTTGTTGTTGAGCATCAGAAGAAG | |
| | AACATCACCAATACAACTTCAAATTTGTCGGTTACTT | |
| | CCGCCAACGATGATCGCGTATACCCAGGTGCTCTTTT | |
| | CCGTGCTGATAAGAATTTGATGGACAATATGCCAAGC | |
| | CTGATTTCTGCAAACCGCGCTCCAATAACGTTGAGCG | |
| | TTGATTTGCCGGGATTCCACGGCGGTAAAGTGCTGTA | |
| | ACTGTTCAGCGCCCAACCAAGAGTTCTGTAACTTCCG | |
| | CAGTGAACGGCTTAGTTTCTAAGTGGAATGCACAATA | |
| | TGGAGCAAGTCATCATGTTGCAGCTCGCATGCAGTAC | |
| | GATTCTGCAAGCGCACAAAGCATGAACCAGCTCAAG | |
| | GCTAAGTTTGGTGCTGATTTTGCCAAGATTGGTGTTC | |
| | CGCTGAAGATTGATTTCGATGCAGTACACAAGGGTGA | |
| | GAAGCAGACTCAAATTGTGAACTTCAAGCAAACTTAC | |
| | TACACCGTAAGCGTTGATGCACCAGATAGCCCAGCA | |
| | GATTTCTTTGCTCCTTGCACTACGCCAGACAGCTTGA | |
| | AGAACCGTATCGATTGAG | |
| pheSmut2_alt | GTTACTCGAGCGTAATGCGTCGTACATACCGCACATT | 32 |
| gblock | AAAATGAACATGTTAGTTTAAATAACGAAGACCAAA | |
| | ACGTATTGTTAAAAATCGCGAAAAATCGCTAGAACTA | |
| | TGCGTTAAGGTTTCACTCAAGGGAGATCCCATATGTT | |
| | GCATAAATCTACGCACAACACTGACCGCAAAGGAGC | |
| | CGTGGCGTCTAGCAAGCCGTTCGACGCTAAAGCTATT | |
| | ACTAAAGTTGTGAAAGAAGGTATAGCCTGCGTACAG | |
| | GCAGCTAAAACAATGGAAGAGTTGAAGGCGGCTAAG | |
| | ACGAAGTATGCCGGCGCTCAATCCGCTATGACACTTG | |
| | CAAGCAAAAGCATTGGATCCTTGCAAGTTGAGGAGA | |
| | AGAAGGAAGCTGGTAAGATTATGTCTGCTTTGCGCGC | |
| | GGATTTTGGCCGCGAGTTCGCTGCAGCCCAAGAGCGC | |
| | ATTAAAGCGATTGAAGAGGCTAATATGCTTCAAAAG | |
| | GAAACGGTAGATATGACATTGCCGATAAACCGCAAA | |
| | CCGCTTGGAGCACGTCATCCGATTGCGCGTATTATTG | |
| | AAGATTTCGAGGACTTCTTCGTGTCTATGGGATGGCA | |
| | AATTTCTGCGGGTCCGGAAGTTGAGACGGAGTGGTTT | |
| | GATTTCGACGCGTTGAATTTCGGTCCAGACCACCCAG | |
| | CTCGTCAGATGCAGGATACCTTCTACGTACAAGGAAA | |
| | TCAGGCCAAAGATGCGGCTGGATTTGTTGGATCGAAT | |
| | ATGGTGCTTCGTACACAGACGTCTTCTGATCAAGTTC | |
| | GTGCATTGATTGAACGTGGTGTTCCACTTTATATTGC | |
| | GAGCCCGGGCAGGGTGTTCAGGACTGACGAACTTGA | |
| | CGCGACTCACACCCCAGTGTTTCATCAATGCGAGGCG | |
| | CTTGCTGTGGATAAGCATCTTTCCATGGCCGACCTTA | |
| | AAGGCGTTCTTGATAGGCTTGCTGTTGCAATGTTCGG | |
| | CCCAGACGCGAAGTCTCGTCTGCGCCCGTCTTACTTT | |
| | CCGTTTAGCGAACCGTCTGCGGAATTGGACCTTTGGT | |
| | TTCCGGACAAAAAAGGAGGACCAGGCTGGATAGAAT | |
| | GGGGTGGATGCGGCATGGTGAACCCAAATGTACTTA | |
| | AATCCGCTGGTCTTGACCCAGACGTTTACACGGGATT | |
| | TGGTTTTGGTGTCGGTCTTGAACGCACACTGTTGCTG | |
| | AGGCATGACATTAACGATATGCATGACTTGGTAGAG | |
| | GGCGACAAACGTTTCTCTGAGCAGTTTGTTATGGGTG | |
| | AGTAGGGTACCTCAG | |
| Δvly gblock | GTCGACTAGTCCAGACTCACTTCTAGGAGCGCCAGAA | 33 |
| | AGCGCTTCTTCTTGAGTCAATGGCGTAGCAGGCTCGT | |
| | CTGGGCGCAAATACGCTTCAAGAGGTACTGGATTTGC | |
| | AGTAGGTGGCACATCTTCGCCAGCAACTTCCTGCACT | |
| | TTGTTAATGGATTCTGCAATGACGTTTAATTCGCCTTG | |
| | CAAGCGACTGATTTCTTCATCGCTTAAAGCAATCTGG | |
| | GACAAAACACCCAAATGCTCAATTTCTTCGCGTGTGA | |
| | ATGTAGGCATAACCTCAACTATATGTGTGATGCGTGA | |
| | CCTTCTTATAGAAGAAAAAAGCAATATTGATAAAAG | |
| | ATTTACACACGTTACTGTAGAAATATTTTCCCAATTTT | |
| | AGAACATGATTGTCGATCCATATTTTCTAGGGAAAAT | |
| | TATGTGTAATATTAAAAAAAATTTTATTAGTGAGACA | |
| | ATAAGCGAAATAACAATTATTGTCACATACAGTTTTC | |
| | ATAAAATTAACGTTATTTGCAGAATATTTAAATATTT | |
| | TAGATATCGCGCTAAATATCGCGATGCTTTTCCACAA | |
| | CGTGGCCAATTGCATACATGACTACGCCCCAAGCAAG | |
| | AACCACCAAGCCTGATTGCCACCATGTAAAAACATAT | |
| | GCGTTTGGTGGAAGCTGTGCACCCGAATTAGGGGAG | |
| | CCGCCCAAGAATTTCTCCACCGCTGTGGCTGGCAAAA | |
| | GCTGTATAAGTATTGAATTCCACTTCGCGAAATTGCT | |
| | TGCAAACATAATAATGCTAAGAACACTAGGCAAAAT | |
| | CATCACGGCTCCAATAACGCACATAATTCCGCCAGCA | |
| | GTTGACTTGCAAATCATGCCAAAGCCGTACGCCATTG | |
| | CTGCTACAACAACCATAATTGCAGGAGAGCCAAGAA | |
| | ACAGCGTAAGAGGCAATTTCCACGCGTTGCTTCCAGA | |
| | TAATCCAGAAGTGTTGTCTCCTATAAAGGCTAATTCT | |
| | GCAGCGCCTAGCGAAACTGCCATTGCCCGGGTATA | |
| Gv pknB_srtE | ATATGAGCTCGCTCTTGGTCTTATCCCAGATATTTTGG | 34 |
| gblock | AAGATGATAAATCTTCTCAACCAGAAGGAACTTTTAC | |
| | AAAACAGCTTCCTAAAGGTGGAGCTAAAGTTTCTTCA | |
| | GGATCTAGAGTAAGCGTATGGTTCTCTGTAGGTCCAC | |
| | AGTCTACTAAGATTCCAGATGTTACTGGTAAATCGCA | |
| | AGATGTAGCACGTAAAGCTCTTGAACGTGCTGGATTT | |
| | AAGATATCTAATGTTCGTGTAGAAGATAGCACGGAA | |
| | GTTAAGAAGAATCATGTTACTCGTACAGATCCTTCTG | |
| | CAGATTCGTTTGCAGACAAGGGGTCTATGGTTACCTT | |
| | ATACATTTCTTCTGGTCTTACAAAGATCCCTGATGGTT | |
| | TAGTTGGACAATCTAAGGATGTTGTAACAAGCGAATT | |
| | GCAAAATCTTGGTTTTACTGTAAATGTTGTTGAGGAA | |
| | GAATCTGATACTGCATCTGAAGGAACTGTGACCAAG | |
| | ATGAACCCATCTTCTGGTGCTGCAGTAAAGCCTCATA | |
| | GCTCTGTAACAGTATATGTTTCTAAAGGAAAGCCTAA | |
| | AGTTGAAGTTCCTCGTTTGGCTGTTGGCACTGTAACTT | |
| | TCAAGCAGGCTAAACAAGTATTGGAAGCAAAAGGCT | |
| | TTAAGGTTGTTGCATCTGATGCATCAGCTAAAGATGA | |
| | TGACATAGTTACTGGAATGTCTGAAAAAGAAGGAGC | |
| | AAAGATTGACAAAGGTTCAACAATCACTTTGACTGTG | |
| | AAATCTGCTACTCCTCCATCGGACCTTACAAAGCCAG | |
| | GTACGGGATTAGATTTGAATAAACCTTCTACTGAAGC | |
| | CCCATCTACATCTACTACAACAACAGATACAGGTTTG | |
| | TAAAAAGCAAAGCATAAAGCCAGTAAGTTTAATAAG | |
| | CTTACTGGCTTTCCATGGTTAGTATTAAGCGTCTGATA | |
| | TGATTATCTTTTGCGTGTCCATTGAGCATGAATGTTGT | |
| | CGCGTTATATGATTGCTCGTTTTTTATTGTCTACTCGG | |
| | ATACGCGAATACGCGCCATGTACGCGTCCGTTCGTGT | |
| | CGGTCGGCTTGGATTTAGACATATGTTATCCAAGTTG | |
| | CATGCGAACAGGCATGGCCAGGCAAGAACCGACACC | |
| | GGTCTCCACCGCGGTGGCGGCCGCACTAGTGGATCCC | |
| | CCGGGCTGCAGGAATTCTGTACACCTAGGATATCAAG | |
| | CTTATCGATACCGTCGACGGCCGGCCTTTATTATTTAT | |
| | TAATCTTATCTACTTACTTATTATCTACTTACCCCTTA | |
| | GTAAGGCGTTGCTGTGTATGCAGACATTTCTCTTAAG | |
| | AATGGTATATTTGCCGAAACAAACGGGTATACCCATT | |
| | GCATAAGTATCATAATGCATATTAGGTAGATAATTAA | |
| | GGAGAGAATTAATCTCACTGGAAGAATTCCAGGCTGT | |
| | AGCCTCATGAGTCCGCCAAAGATGCTGAATTCAGGCT | |
| | TTGGCTTTAGACCTGCTTTAATCTCGCGTCTTAATGGC | |
| | CACTGCCATGCAATAGCTCCAGCAGCAAAGATAATC | |
| | AAATAAACTATCAGCGCGCCAATCATAAGCGGAACA | |
| | AGCGAATCGAGATGAGAGATCAAAGATTGTTGCTCG | |
| | TTATTAACAAACTTAACTTTTCCGTTTTCATCTAAAGT | |
| | AGAAAGCTCTTTTGGAATACCGTCAGAAACTTTAGCC | |
| | CAATAGTCAAGCTCTCCAAAGCTAACAAAGCGGAAC | |
| | TTAGGTGTAGAGAACTTAGGTTCGCATGTAATAATGG | |
| | TGATCATGCGTTTCTTTGGCTCTTTACTATGGTTTATT | |
| | GGATCTGGGTCAAGAACCTCAACCTGTTCTGGTCTAA | |
| | CAATCTTGTGAGTAATGTATTTGTAAACGAACCAATA | |
| | ATCTTTAGTTCGAAGAATAATAGAATCTCCCTTTTGG | |
| | AACCTATCAACATCCGCAAGAGGCTGACCATAACCAT | |
| | TGCGGTGACCAATAATTGTAATGTTTCCGATTTCTCC | |
| | AGGCAACTCAGTTTTAGGATAATGACCTAATCCTCGA | |
| | CGATTAAGTATCTCCATATCTACACCTTCAATAACAT | |
| | TACGCTCCCATTGATCACCGAAGCGTGGAATATAGAT | |
| | TCGTGCTCGAGTATC | |

*Restriction sites are underlined.
Bolded nucleotides in oligos and gblocks represent changes to the wild-type sequence.

Preparation of electrocompetent cells: G. vaginalis ATCC 14018 was streaked from a frozen stock onto an HBT agar plate and grown at 37 °C with 5% CO₂ overnight (24 hr). Colonies were swabbed from the HBT plate into 6-12 ml BHI-YDS medium, and the density was adjusted to an OD₆₀₀ of 0.05. The culture was grown in a 15-ml conical tube held stationary overnight (18 hr). The overnight culture was diluted into 30-45 ml of fresh BHI-YDS medium to an OD₆₀₀ of 0.1-0.15 and incubated statically at 37 °C with 5% CO₂. For initial transformation experiments, cells were harvested when the OD₆₀₀ reached 0.35. In later transformations, cultures were grown to an OD₆₀₀ of 0.25-0.3, then D-cycloserine (DCS) was added to a concentration of 250 μg/ml, and the culture was incubated another 1.5 hr before harvesting the cells. Cultures were placed on ice for 15 min and the cells were divided between two 50-ml Oak Ridge tubes and harvested by centrifugation for 10 min at 8000 rpm in a Sorvall RC6+ centrifuge at 4 °C. The supernatant was removed, and each cell pellet was resuspended in 1 ml of ice-cold 10% glycerol in water and transferred to two 1.5-ml microfuge tubes. Using 1.5-ml microfuge tubes rather than Oakridge tubes at this step minimized cell loss. The cell suspension was centrifuged for 5 min at 8000 rpm at 4 °C in a refrigerated centrifuge. The supernatant was discarded, and the cells were washed twice more. After the first centrifugation, the cell pellet was sticky and difficult to resuspend. However, with the successive washes, the pellet became easier to resuspend and formed a uniform suspension. The final cell pellets were combined and resuspended in a volume of ice-cold 10% glycerol that was 1/100 of the starting culture volume. Aliquots of 80 μl were placed in 1.5-ml microcentrifuge tubes on ice or stored at −80 °C.

Transformation protocol: Oligo DNA (10 μg), or plasmid DNA (1.5-2 μg), was mixed with 80 μl of electrocompetent cells and incubated on ice for 5 min. Cells without DNA were included as a negative control. The cell suspensions were transferred to pre-chilled 0.1-cm gap cuvettes (BioRad) and electroporation was performed, initially on a BioRad Micropulser unit with 2.2 kV, and later on a BioRad Gene Pulser or BioRad Gene Pulser Xcell™ unit with parameters of 200 Ω, 25 μF, and 2.2 kV. Time constants were recorded following the electrical pulse. Cells were immediately resuspended in 4 ml of pre-warmed BHI-YDS medium and allowed to recover statically overnight (18-20 hr) at 37 °C with 5% CO₂. Serial dilutions of the overnight cell suspensions were spotted on BHIFF agar to obtain total CFU/ml. Aliquots of the recovered cells were spread on BHI-YDS agar supplemented with the appropriate antibiotic. For transformations with oligos, 50 μl or 100 μl aliquots were plated; for transformations with plasmids, 1-ml aliquots that were concentrated 10-fold were plated. Plates were incubated at 37 °C with 5% CO₂ for two days. The transformation frequency was calculated as the antibiotic resistant CFU/ml divided by the total CFU/ml. Putative transformants were restreaked to BHI-YDS plates containing the appropriate antibiotic. Putative rpsL transformants were sequenced to verify incorporation of the mutation present on the oligo, while isolates transformed with plasmids were analyzed by PCR to verify the presence of the plasmid at the target locus in the chromosome.

Methylation of plasmids: Plasmids were isolated from E. coli with the QIAGEN Miniprep Kit, then methylated with HaeIII methyltransferase (NEB) according to the manufacturer's instructions. Methylated plasmid was purified from the reaction mixture with the QIAquick® PCR Cleanup Kit (Qiagen) or the Monarch® DNA and PCR Cleanup Kit (NEB), according to the manufacturer's instructions. Approximately 1.5-2 μg of methylated plasmid was concentrated by centrifugation under vacuum in a speed vac to 1-2 μl in preparation for electroporation.

Construction of insertion-duplication mutants: To construct an insertion-duplication (ID) plasmid for interrupting the gene for vaginolysin (vly), an approximately 0.5-kb internal fragment of vly was amplified by PCR from G. vaginalis strain ATCC 14018 with primers vly_SacI-F and Gv_abc-R. The PCR product was digested with SacI and ClaI and ligated into pIDN1 that had been digested with the same enzymes and dephosphorylated. The ligation mixture was transformed into chemically competent E. coli, and transformants were selected on LB plates containing Erm. The recombinant plasmid, pAKK134, was treated with HaeIII methyltransferase (NEB), and approximately 1.5-2 μg of methylated plasmid was electroporated into ATCC 14018 electrocompetent cells. Controls included cells without DNA, and cells with 10 μg of the 44-nt oligo rpsL4mut. Following overnight (18-20 hr) recovery, the negative control and transformed cultures were plated on selective agar. After 2 days of incubation at 37 °C with 5% CO₂, colonies were enumerated. Colonies from the plasmid transformation plates were restreaked to the same medium to confirm resistance to erythromycin. Erm^{R} isolates were screened by colony PCR with a primer that annealed to the genome (vly-F), along with a primer that annealed to the vector (pmob-R) to identify transformants that had the plasmid integrated at the correct locus. Three vly insertional mutants, AKK107-1/2/3, were characterized for hemolytic activity.

An insertion-duplication plasmid was constructed to interrupt the gene encoding the HaeIII-like endonuclease gene in ATCC 14018. A 447-bp internal fragment of the haeIIIR gene from ATCC 14018 was PCR amplified with primers Gv_haeIII_SacI-F and Gv_haeIII_ClaI-R, and the digested PCR product was ligated into the SacI/ClaI sites of pIDN1. This construct, pAKK139, was treated with HaeIII methyltransferase and transformed into ATCC 14018 as described for pAKK134. PCR analysis of Erm^{R} transformants with a primer that annealed to the genome (haeIII_screen-F) and one that annealed to the vector (pmob-R) confirmed plasmid integration at haeIIIR.

VLY hemolysis assay: Gardnerella strains were streaked onto HBT plates from frozen stocks and grown overnight at 37 °C with 5% CO₂. Colonies were swabbed into BHI-YDS medium, and suspensions were adjusted to an OD₆₀₀ of 0.05. Replicates of 200 μl of inoculum were placed in the wells of a 96-well tissue culture-treated plate and grown statically at 37 °C with 5% CO₂ for 21-24 hr. The initial and final ODs were measured as an indication of growth, and culture supernatant from triplicate wells was combined. Cells were removed by centrifugation, and 500 μl of supernatant was concentrated with a 30-kD cut-off column (Amicon Ultra 0.5 ml). Samples were stored at −80 °C prior to analysis. Single donor human whole blood was purchased from Innovative Research Inc. Erythrocytes were collected by centrifugation (500×g for 5 min), plasma was removed, and the cells were washed three times with sterile PBS. A 1% solution of erythrocytes was prepared. Concentrated supernatant was serially diluted with PBS, and 100 μl of each dilution was mixed with 100 μl of the 1% erythrocyte suspension in a V-bottomed 96-well plate. Positive and negative controls for hemolysis were a 0.1% solution of Triton™ X-100 (Tx) and PBS, respectively. The plate was incubated for 30 min at 37 °C with 5% CO₂ and then centrifuged for 10 min at 2000 rpm to pellet intact erythrocytes. Supernatant was transferred to duplicate wells of a flat-bottomed 96-well plate and the absorbance (A) at OD₄₁₅ was measured. Percent hemolysis was calculated as (Aₛₐₘₚₗₑ−A_{PBS})/(A_{Tx}−A_{PBS})×100.

LacZ insertion-duplication constructs: Four internal fragments of the beta-galactosidase gene (lacZ) from ATCC 14018 ranging in size from 0.2-1.8 kb were cloned into pDIN1. The 0.2-kb ‘lacZ’ fragment was ordered as a gblock from IDT (Integrated DNA Technologies). The 0.5-, 1- and 1.8-kb fragments were PCR-amplified from genomic DNA of ATCC 14018 with Gv_lacZ_SacI-F paired with Gv_lacZ(0.5 kb)_XhoI-R, Gv_lacZ(1 kb)_XhoI-R, or Gv_lacZ-R3, respectively. The gblock and PCR products were digested with SacI and XhoI, then cloned into the SacI/XhoI sites of pIDN1, resulting in constructs pAKK144, pAKK145, pAKK146, and pAKK148, which contain successively larger ‘lacZ’ inserts. The plasmids were treated with HaeIII methyltransferase and transformed into ATCC 14018 competent cells, resulting in insertion-duplication mutants AKK112, AKK113, AKK114, and AKK15, respectively. Integration at lacZ was confirmed by the white colony phenotype on BHI-YDS agar supplemented with 40 μg/ml of X-Gal.

Plasmid excision assay: Strains AKK112-AKK115 were streaked out onto HBT agar and grown overnight at 37 °C with 5% CO₂. Colonies were swabbed from 24 hr plates into BHI-YDS medium and 6 ml of bacterial suspension was adjusted to an OD₆₀₀ of 0.05. Liquid cultures were grown stationary at 37 °C with 5% CO₂ for 18-20 hr. Serial dilutions were prepared and spread on BHI-YDS agar containing 40 μg/ml X-Gal, while spot dilutions were plated on BHI-YDS agar to obtain total CFU/ml. After overnight incubation, blue colonies on the plates with X-Gal were enumerated and the frequency of plasmid excision was calculated as blue CFU/ml divided by total CFU/ml.

Construction of positive-negative selection plasmids for creating unmarked mutations: To create unmarked mutations in G. vaginalis, an allelic exchange vector was constructed that contained a counter-selectable marker. In E. coli, PheS with the double mutation T251S A294G incorporates the toxic analog 4-chloro-DL-phenylalanine (4CP) into proteins, resulting in cell death. Alignment of the protein sequences of PheS from ATCC 14018 and E. coli K12 showed that ATCC 14018 contained the conserved Thr and Ala residues. Therefore, a gblock was designed containing the pheS from ATCC 14018 with the double mutation (T286S A333G), designated phe_{Smut2}. A strong promoter (from the gap gene in ATCC 14018) was incorporated to drive expression of phe_{Smut2}. The gblock was digested with ClaI and XhoI and cloned into the ClaI/XhoI sites of pIDN1, creating pAKK163. Next a gblock with a 0.7-kb internal fragment of the vaginolysin gene that contained a nonsense mutation (E155*; GAA to TAA change) near the middle was digested with SacI and SmaI and ligated into the SacI/SmaI sites of pAKK163, resulting in plasmid pAKK165.

Another version of the positive-negative selection plasmid, pAKK174, was constructed that contained the phe_{Smut2} gene with alternative codons (designated pheS_{mut2_alt}) to reduce the possibility of integration at the pheS locus. The codon optimization tool by IDT was used to convert the phe_{Smut2} codons from G. vaginalis to codons optimized for another member of the Bifidobacteriaceae family, Bifidobacterium longum. After the optimization, codons that appeared to be rare in the original ATCC 14018 pheS sequence were manually changed to more representative codons. Next a gblock was designed to create a complete deletion in vly. DNA fragments upstream (484 bp) and downstream (491 bp) of vly were fused to create a 1935-bp deletion that removes the entire vly coding sequence (1551 bp), in addition to some sequence flanking either side of the gene (277 bp and 107 bp, respectively). The Δvly gblock was digested with SpeI and SmaI and cloned into the SpeI/SmaI sites of pAKK174, generating pAKK176.

Creation of unmarked vly mutants using positive-negative selection constructs: A two-step method of gene replacement was used to create unmarked mutations in vly with pAKK165 (nonsense mutation) or pAKK176 (full deletion). First, ATCC 14018 was transformed with the methylated plasmid, and integration via a single crossover event was selected for by plating on medium with Erm. Integration at or near vly was confirmed by PCR analysis. Second, after growth of the integrant on medium without Erm, plasmid loss due to a second recombinational event was selected for by plating on BHI-YDS medium supplemented with 10% FBS and 1 mM 4CP. Isolates that have excised the plasmid do not incorporate the toxic 4CP into proteins and are therefore able to grow in its presence. If the second crossover occurs on the same side of the mutation that the first crossover occurred on, the wild-type gene is restored. However, if the second crossover occurs on the opposite side of the mutation, the mutation is incorporated into the genome. After 2 days, colonies were patched onto HBT for phenotypic screening. Sequencing confirmed the presence of the point mutation in or deletion of vly.

Cervix infections: Human cervix samples were obtained from consented donors undergoing hysterectomies through the National Disease Research Interchange (NDRI). Donors were 50 years of age or younger and had not undergone chemotherapy or radiation treatment. Tissues were shipped overnight on ice in Dulbecco's Modified Eagles Medium (DMEM). On the day of arrival, endocervical and ectocervical regions were processed into 3-mm biopsy punches. Tissue punches were allowed to recover overnight and were maintained in cervical tissue culture medium (CTCM: CMRL-1600 medium, 0.1 μg/ml hydrocortisone 21-hemisuccinate sodium, 1 μg/ml bovine insulin, 2 mM L-glutamine, 5% heat-inactivated FBS) with penicillin and streptomycin at 37 °C with 5% CO₂ until the infection experiment. This work was determined to be exempt as human subjects research by the University of Wisconsin Health Sciences IRB as NDRI codes each tissue sample and does not label or provide identifying information.

Endocervical and ectocervical punches were washed with sterile PBS and moved to fresh CTCM. ATCC 14018 and AKK124 were streaked onto HBT from freezer stocks and incubated overnight at 37 °C with 5% CO₂. The strains were restreaked onto HBT and incubated overnight. Bacteria were swabbed from the plates into PBS, washed in CTCM, and added at a final OD₆₀₀ of 0.2 (~1×10⁸ CFU/ml) in 1 ml of CTCM to cervix tissue in a 24-well plate. Two replicates per treatment were included. Plates were incubated at 37 °C for 24 hr. 50 μl aliquots of supernatant were removed after 0, 12, and 24 hr of incubation, mixed with the Promega LDH Storage Buffer (200 mM Tris-HCl (pH 7.3), 10% Glycerol, 1% BSA), and stored at −20 °C until they were analyzed for lactate dehydrogenase activity (Promega LDH-Glo™ Cytotoxicity Assay). The activity of LDH in the supernatant samples was measured using the bioluminescent assay according to the manufacturer's protocol. The infections were repeated with at least three separate human donor tissues.

Construction of complementation plasmid for G. vaginalis: A region in ATCC 14018 was identified that had convergent genes (pknB and srtE) that had high nucleotide conservation between clade 1 strains and thus could serve as a complementation site in G. vaginalis. A gblock was designed that had 0.8-0.9-kb fragments containing the 5′ ends of the pknB and srtE, with a multiple cloning site (MCS) between the two fragments. The promoter from the 14018 rpsB gene was included to direct constitutive expression of the gene of interest. Inclusion of unique restriction sites on either side of the rpsB promoter (NcoI and AgeI) allows for replacement with a different promoter. The gblock had SacI and XhoI sites added to the ends to enable cloning into these same sites in pAKK182, an E. coli plasmid that contains a spectinomycin resistance gene from Enterococcus faecalis, that we found provided resistance in G. vaginalis. The ermC gene was subsequently cloned into the SalI site within the gblock, resulting in the complementation construct pAKK186.

The vly gene from 14018 was directionally cloned into the AgeI and BsrGI sites within the MCS, producing pAKK187. pAKK187 was methylated with HaeIII methyltransferase and electroporated into AKK132 (Δvly), selecting for Erm^{R} transformants. Erm^{R} isolates were patched to plates containing spectinomycin to identify those that had integrated the full plasmid by single-crossover recombination rather than the desired transformants that had integrated only the region between pknB and srtE via double-crossover recombination. Some isolates were resistant to both Erm and Spec, while others were resistant to Erm only, suggesting these isolates had undergone a double crossover. PCR analysis was conducted to confirm that the Erm^{R} Spec^{S} isolates were double recombinants.

Creation of nanH3 deletion mutant in Gardnerella 3336: A construct was made that contained nanH3 from Gardnerella strain 3336 with a large in-frame deletion spanning amino acids 6-783 of the 812-amino acid protein. Two DNA fragments of approximately 0.8 kb were PCR-amplified from 3336. One fragment contained upstream flanking sequence and the first 5 codons of nanH3, followed by a SpeI site; the other fragment had a SpeI site followed by the last 30 codons of nanH3 and downstream flanking sequence. The fragments were generated by PCR with primer pairs nanH3_up_SacI-F/nanH3_up_SpeI-R and nanH3_down_SpeI-F/nanH3_down_XhoI-R. The fragments were sequentially cloned into pAKK174 which resulted in construct pAKK191; the insert contained the ΔnanH3 allele with a SpeI site inserted in-frame between the two fragments. Strain 3336 lacks the gene for the HaeIII-like endonuclease, so pAKK191 was not treated with the HaeIII methyltransferase prior to electroporation. Competent cells of 3336 were prepared with DCS treatment as described for ATCC 14018, and 2 μg of pAKK191 was electroporated with the same parameters that were used for ATCC 14018. Erm^{R} colonies were obtained at a frequency of 1.6×10⁻⁶. An Erm^{R} integrant, AKK140, was grown overnight without selection and plated on medium containing 1 mM 4-CP to select for recombinants that had lost the plasmid insertion. A PCR screen of seven recombinants showed that four had the ΔnanH3 allele. The mutant was designated AKK141. DNA sequencing confirmed the presence of the in-frame deletion.

Sialidase activity of 3336 and nanH3 deletion mutant: Strains 3336 and AKK141 (ΔnanH3) were grown overnight on HBT media. Colonies were swabbed into PBS, and the bacterial suspensions were adjusted to an OD₆₀₀ of 0.5. Cells from 1.5 ml of each suspension were harvested by centrifugation and resuspended in 0.75 ml of 0.1 M sodium phosphate buffer, pH 5.8. The suspensions were sonicated on ice for 30 sec (1 sec on/1 sec off) at 40% amplitude to lyse the cells. Cellular debris was removed by centrifugation, and 100 μl of supernatant were transferred to a new microfuge tube. 5 μl of 10 mM X-NeuNAc substrate (APExBIO) was added to the samples or to buffer and incubated overnight at 37 °C with 5% CO₂. The samples were transferred to a flat-bottomed 96-well plate, and the absorbance at OD₆₁₅ was measured on a BioTek Synergy HT plate reader. The blank measurement was subtracted from the samples. Three independent assays were performed.

Statistical analysis: The F-test was used to compare variances between two groups. Statistical analysis was performed using a Student's t test for data with equal variance or a Welch's t test for data with unequal variance. P values of less than 0.05 were considered statistically significant.

### Example 1: Preparation of Electrocompetent Cells and Transformation of G. vaginalis with Oligonucleotides

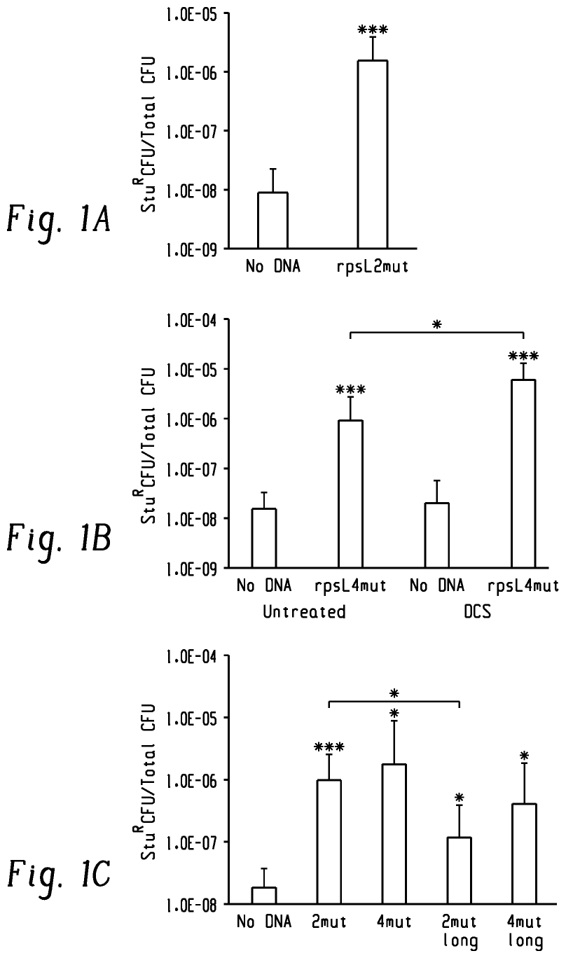

To determine if DNA could be transformed into G. vaginalis by electroporation, oligo-mediated recombination was attempted. G. vaginalis ATCC 14018 was selected for these experiments, and the large colony variant was used due to its robust growth. Cells were grown in liquid culture to early log-phase (OD₆₀₀~0.35), chilled on ice, harvested by centrifugation, and washed with 10% glycerol. The final cell density in this suspension was approximately 2.0×10⁹ CFU/ml. For oligo-mediated recombination, a mutagenic 44-base oligonucleotide was designed that would introduce a dinucleotide substitution (AA to CG) in the rpsL gene, conferring streptomycin resistance. In other bacteria, transformation efficiency can be increased by using levels of oligonucleotides that saturate the host exonucleases. Thus, a relatively high concentration of oligo was used; G. vaginalis cells were mixed with 10 μg of oligo in ice-cold cuvettes and electroporated at 2.2 kV. Cells were allowed to recover overnight in supplemented brain heart infusion medium (BHI-YDS) before plating on agar medium containing streptomycin. After two days, Str^{R} colonies were obtained at frequencies of approximately 10⁻⁶ Str^{R} CFU/total CFU (FIG. 1A). Sequencing of the rpsL gene in Str^{R} isolates showed that the 2-base mutation had been incorporated.

Once oligo-mediated recombination had been achieved, a variety of parameters were altered to determine the impact on transformation efficiency. Harvesting cells at a later point in growth (OD₆₀₀~0.5) resulted in a 4-fold reduction in the number of transformants compared to cells from early log-phase. Reducing the voltage of electroporation from 2.2 kV to 1.8 kV resulted in half as many recombinants. Increasing the amount of oligo from 10 μg to 50 μg did not impact the number of transformants, suggesting that 10 μg was a saturating amount of oligonucleotide. No Str^{R} colonies were obtained if the electroporation was done with a room temperature cuvette. Also, no transformants were recovered if the cells were allowed to recover for only 4 hours. Using frozen and thawed cells, rather than freshly prepared cells, reduced efficiency by one third.

### Example 2: Growth of G. vaginalis with D-Cycloserine Increases Transformation Efficiency

It was investigated whether growing the cells in the presence of potential cell wall-weakening compounds could enhance the transformation frequency. ATCC 14018 was grown in the presence of glycine, Tween™ 80, lysozyme, penicillin, DTT, or lithium acetate, and electroporated with an rpsL oligo containing 4 base changes (TAAG to ACGC), conferring streptomycin resistance. None of these treatments increased the efficiency of transformation with the rpsL oligo. Growing cells in the presence of sucrose and washing with a sucrose-based buffer was also tried, but again no increase in transformation frequency was observed. However, addition of a sub-lethal concentration of D-cycloserine (DCS) to the culture had a positive effect on transformation efficiency. ATCC 14018 was grown to an OD₆₀₀=0.2-0.25, at which time DCS was added to a concentration of 250 μg/ml, and the culture was grown for another 1.5 hours. Following growth with DCS, cultures exhibited a reduced OD₆₀₀ compared to untreated cultures. Electroporation of DCS-treated cells with the rpsL oligo resulted in a 6-fold increase in Str^{R} frequency compared to non-treated cells (FIG. 1i). Therefore, this treatment was included in subsequent competent cell preparations. DCS is an antibiotic that inhibits alanine racemase and D-Ala D-Ala ligase, both of which are involved in peptidoglycan synthesis. It is likely that treatment of G. vaginalis with DCS reduces the extent of crosslinking between peptidoglycan strands and thereby weakens the barrier function of the cell envelope.

### Example 3: Longer Oligos do not Result in Higher Transformation Frequencies

In E. coli, the DNA mismatch repair system can correct mismatches which naturally occur during replication, but it also represents a barrier to the introduction of point mutations. However, mismatch repair can be overcome by introducing consecutive mutations or C-C mismatches. Transformation frequency with the 44-base rpsL oligo containing mismatches in 4 consecutive nucleotides (TAAG to ACGC) was compared to the original oligo that has 2 nucleotide changes (AA to CG). The 44-base oligo with the 4-base mutation resulted in a 1.8-fold higher average transformation frequency compared to the oligo with the 2-base mutation, but this apparent difference was not statistically significant (FIG. 1C). Two longer oligos, both 84 nucleotides, that contained the same 2-base or 4-base mutations, were tested to determine whether a longer homologous region would increase transformation efficiency. However, electroporation with the longer oligo resulted in an 8-fold lower transformation frequency compared to the corresponding shorter oligo for the 2-base mutation oligos in three independent assays (FIG. 1C). A similar trend was seen for the 4-base mutation oligos, but the difference was smaller and did not reach the level of significance.

### Example 4: Creation of a Vly (Vaginolysin) Mutant Using Circular, Methylated DNA

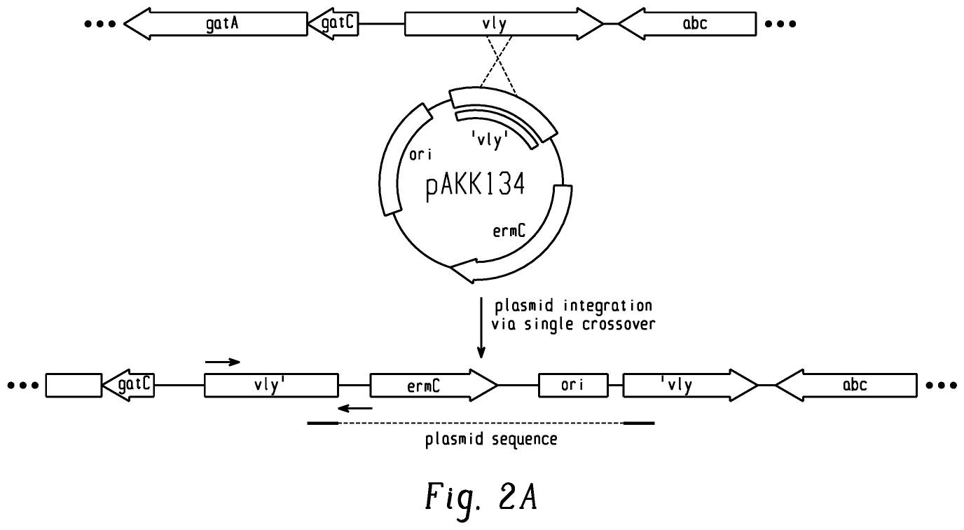

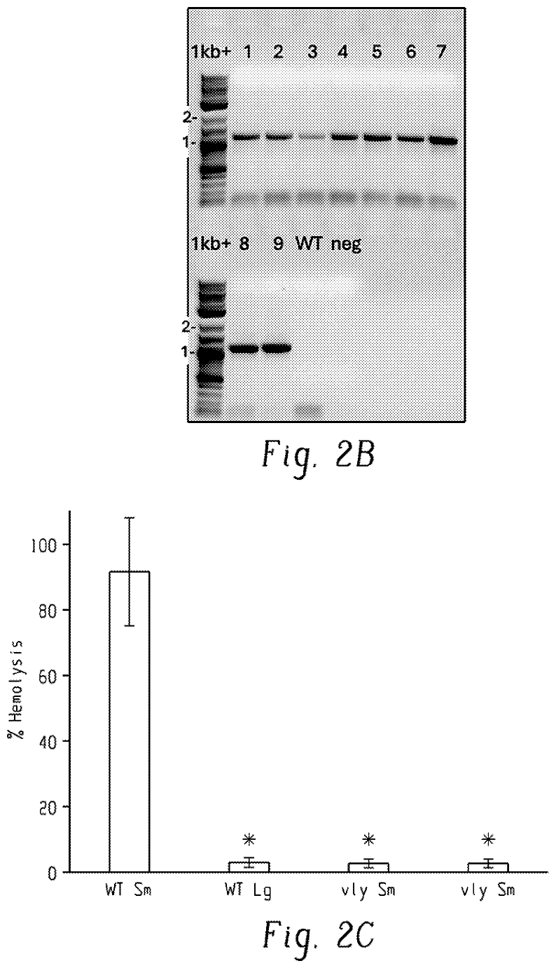

After successful transformation with the rpsL oligos, transforming various larger pieces of DNA into G. vaginalis was attempted. Chromosomal DNA from Gardnerella strains that naturally carry a tetM marker was tried. Linear constructs which we built carrying an ermC or tetM marker were inserted into the vly (vaginolysin) gene which had been cloned from strain ATCC 14018. None of these forms of DNA worked for transformation. Next, it was considered that small plasmids might work for transformation. No plasmids have been isolated from Gardnerella spp. Therefore, an insertion-duplication mutant was attempted by integration of an E. coli plasmid into the G. vaginalis chromosome, selecting for erythromycin resistance encoded on the plasmid. An approximately 0.5 kb fragment internal to the vaginolysin gene was cloned into E. coli plasmid pIDN1, and 2 μg of resulting plasmid pAKK134 was electroporated into ATCC 14018 (FIG. 2A). The first attempts to obtain plasmid transformants were unsuccessful. Erm^{R} isolates were obtained at a frequency no different from a no-DNA control, and PCR analysis determined that these isolates did not contain the plasmid construct. However, the report of a HaeIII-like type II restriction-modification (RM) system in ATCC 14018 that cleaves at unmethylated GGCC sequences suggested that this endonuclease activity might be preventing plasmid transformation. To avoid restriction of plasmid DNA, pAKK134 was treated with HaeIII methyltransferase and repeated the electroporation. Erm^{R} colonies were obtained at a frequency of 1.6×10⁻⁷, over 50-fold higher than the frequency of spontaneous Erm^{R} colonies obtained in the negative control (2.8×10⁻⁹). PCR analysis with one primer that anneals to the chromosome and one that anneals to the plasmid confirmed that nine Erm^{R} isolates from the plasmid transformation all contained pAKK134 integrated at vly (FIG. 2B). A hemolysis assay with concentrated cell supernatants from the wild type or one of the insertion-duplication mutants showed that the mutant was reduced in hemolytic activity compared to the wildtype (FIG. 2C). The small colony variant (Sm) of wild-type strain ATCC 14018 produces large amounts of vaginolysin, while the large colony variant (Lg) produces much less (FIG. 2C). The vly mutant strain AKK107 exhibited only background levels of hemolytic activity against human red blood cells whether the Sm or Lg variant was used (FIG. 2C).

### Example 5: a haeIIIR Mutant is Transformable with Unmethylated Plasmid

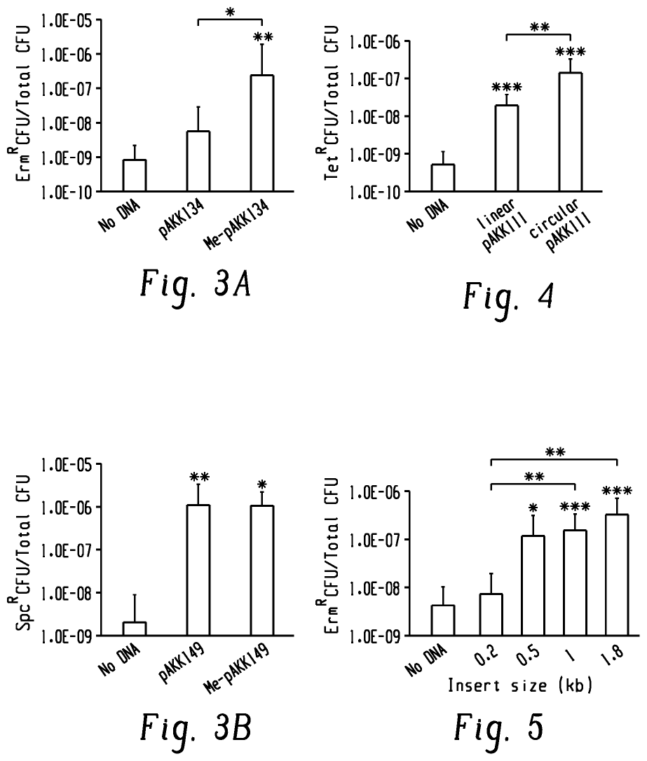

In subsequent transformations with untreated pAKK134 and methylated pAKK134, sometimes transformants were obtained with untreated plasmid, but the frequency with methylated plasmid was on average 37-fold higher than with untreated plasmid (FIG. 3A). An insertion-duplication mutant was constructed to knock out the HaeIII-like endonuclease gene in ATCC 14018 to examine whether transformation of the mutant with an untreated plasmid could be obtained at a frequency similar to that with a methylated plasmid. ATCC 14018 was transformed with Me-pAKK139 (‘haeIIIR’), resulting in Erm^{R} insertion-duplication mutant AKK110. AKK110 was transformed with methylated or untreated plasmid pAKK149, which is identical to pAKK134 but with the ermC replaced by aad9, encoding spectinomycin adenyltransferase. Transformation of AKK110 with untreated and HaeIII-methylated pAKK149 resulted in Spc^{R} colonies at a relatively high frequency for this procedure, approximately 1×10⁻⁶ for both conditions (FIG. 3B). The similar frequencies for both methylated and untreated plasmids indicates that the HaeIII-like restriction-modification system was inhibiting plasmid transformation in wild-type ATCC 14018.

### Example 6: Circular DNA Constructs Transform Better than Linear Constructs

To determine if the initial attempts at transformation might have failed due to using linear DNA, transformation frequencies were compared for constructs that were either circular or linearized plasmids. Plasmid pAKK111 contains 1.9 kb of G. vaginalis chromosomal DNA with a tetM marker inserted near the middle of the cloned region. The tetM gene is inserted between 984 bp of vly region DNA and 912 bp of the downstream gene, which encodes an ABC transporter. Linear pAKK111 was produced by digestion with restriction enzyme SphI, which cuts in the plasmid backbone, outside the region homologous to the chromosome. Tetracycline resistant transformants were obtained at a 7-fold higher frequency with the circular plasmid as compared to the linearized plasmid (FIG. 4). These data suggest that linear DNA may be more readily degraded than circular DNA prior to recombination or that single-crossover recombination occurs at higher frequency than double-crossover recombination.

### Example 7: Frequency of Plasmid Integration and Excision Correlates with Length of Homologous DNA

To examine how the frequency of plasmid integration might vary with the length of homologous DNA, a series of constructs that contained an internal fragment of G. vaginalis lacZ that was 0.2-, 0.55-, 1-, or 1.8-kb in length was designed. Plasmid integrants that interrupted the lacZ gene were selected for using erythromycin resistance encoded on the plasmid. Transformation frequency correlated with the size of the insert, ranging from an average of 5.0×10⁻⁹ with the 0.2-kb insert to 2.8×10⁻⁷ with a 1.8-kb insert (FIG. 5). Plasmid integrant colonies appeared white on media containing X-Gal, whereas the wild-type colonies were blue. To determine how stable the insertions were, the frequency blue colonies arose after overnight growth of the mutants in non-selective liquid media was determined. Restoration of the wild-type lacZ gene would occur in isolates that underwent a second recombination that excised the plasmid. The frequency of blue colonies varied inversely with the size of the duplication, ranging from an average of 2.3×10⁻⁵ blue colonies/total colonies with the 0.2-kb insert to 4.8×10⁻³ blue colonies/total colonies with the 1.8-kb insert. This is likely an overestimation of the excision frequency since excision occurring in colonies during early growth on the plate would also generate blue colonies. Nevertheless, given the relatively high frequency of plasmid excision in insertion-duplication mutants, a method that would allow construction of unmarked gene deletions and point mutations was sought.

### Example 8: Construction of a Positive/Negative Selection Plasmid

In order to introduce unmarked mutations into Gardnerella, an insertion plasmid was developed that contains the ermC gene for selection of plasmid integration (positive selection), and a mutated pheS gene for the selection of plasmid loss (negative selection). Use of this positive-negative selection vector allows one to change specific bases in a gene or delete a specific gene using a two-step procedure. pheS containing a double substitution was used as a counter-selectable marker. The pheS gene encodes the a subunit of phenylalanine-tRNA synthetase. In E. coli, PheS with an A294G mutation misincorporates the toxic phenylalanine analog 4-chloro-DL-phenylalanine (4CP) into proteins, leading to cell death. An improved pheS allele containing two mutations, T251S A294G, was engineered which allowed selection on rich media and with lower concentrations of 4CP. Alignment of the PheS proteins from E. coli and ATCC 14018 revealed that ATCC 14018 contained the conserved Thr and Ala residues present in E. coli. Therefore, a plasmid was constructed containing ATCC 14018 pheS with the double substitution (pheSₘᵤₜ₂), preceded by the G. vaginalis promoter for the gene encoding glyceraldehyde-3-phosphate dehydrogenase (P_{gap}), which would ensure a high level of expression of pheSₘᵤₜ₂. This pheSₘᵤₜ₂ was cloned into plasmid pIDN1, resulting in positive/negative selection plasmid pAKK163.

### Example 9: Construction and Characterization of Vly Point Mutation Strain

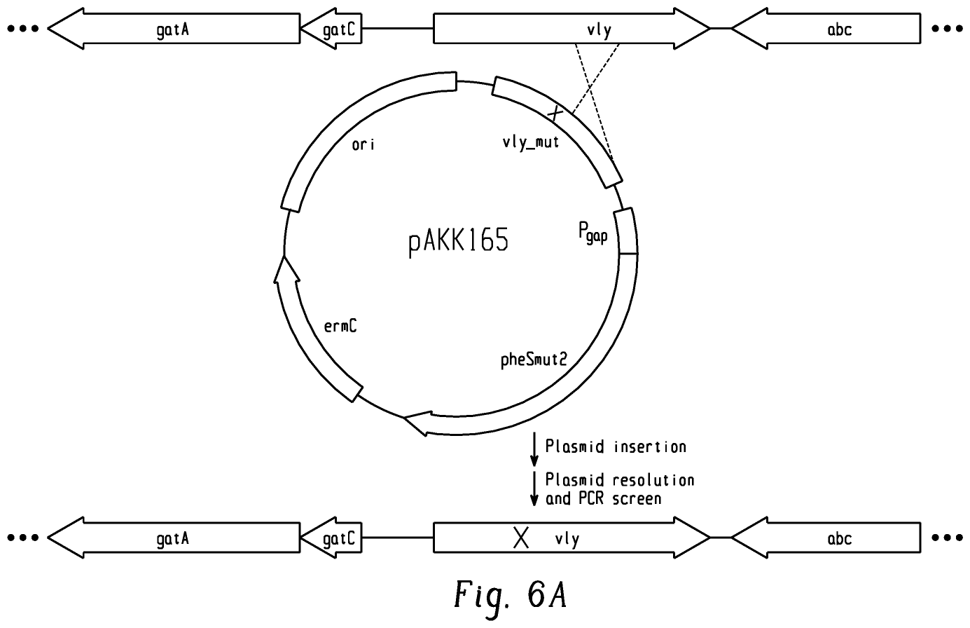

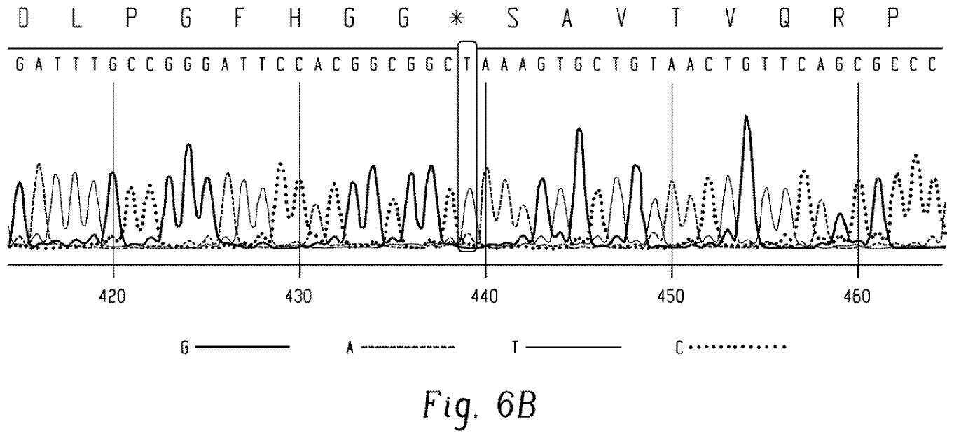

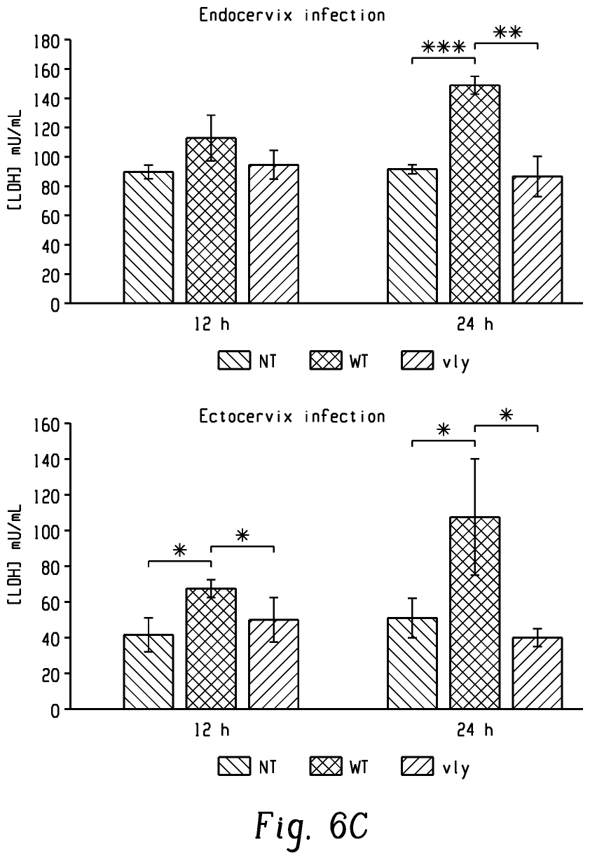

To test whether pAKK163 could be used to create an unmarked mutation, a point mutation was constructed in vly in ATCC 14018. Aa 0.7-kb fragment of the vaginolysin gene that spanned amino acids 39-271 and contained a nonsense mutation at E155 (GAA to TAA) was cloned, resulting in plasmid pAKK165 (FIG. 6A). Following electroporation of the methylated plasmid into ATCC 14018, Erm^{R} transformants were obtained that had integrated the plasmid at vly, and they exhibited diminished hemolysis on HBT agar. The integrant was grown on medium without Erm to allow for a second recombination event and loss of the plasmid. Dilutions of the culture were plated on medium with and without 1 mM 4CP, and 4CPR colonies were obtained at a frequency of 4.7×10⁻⁴. Ninety-three percent of the colonies were also sensitive to Erm, demonstrating that the pheSₘᵤₜ₂ provided efficient counter selection against the plasmid. Colonies from the 4CP plate were patched to HBT agar to screen for recombinants that had maintained the nonsense mutation and appeared nonhemolytic after overnight growth. Approximately 50% of the patches appeared reduced in hemolysis compared to the wild type. Sequencing of the vly gene in a nonhemolytic isolate (AKK124) confirmed the presence of the nonsense mutation (FIG. 6B).

To quantify the cytotoxicity of the wild type and vly point mutant on cervical cells, human ectocervical and endocervical tissue explants were infected with the bacteria and the level of lactate dehydrogenase (a cytosolic enzyme that is released when cells are damaged) was measured. The LDH level from the wild type-infected tissues increased over time and exhibited statistically higher LDH levels than the mutant after 12 and 24 hr of infection of the ectocervical tissue (FIG. 6C, right panel), and after 24 hr infection of the endocervical tissue (FIG. 6C, left panel). The LDH activity measured for the vly mutant was no different from untreated tissue at 12 or 24 hr (FIG. 6C).

### Example 10: Construction of Vly Deletion Strain

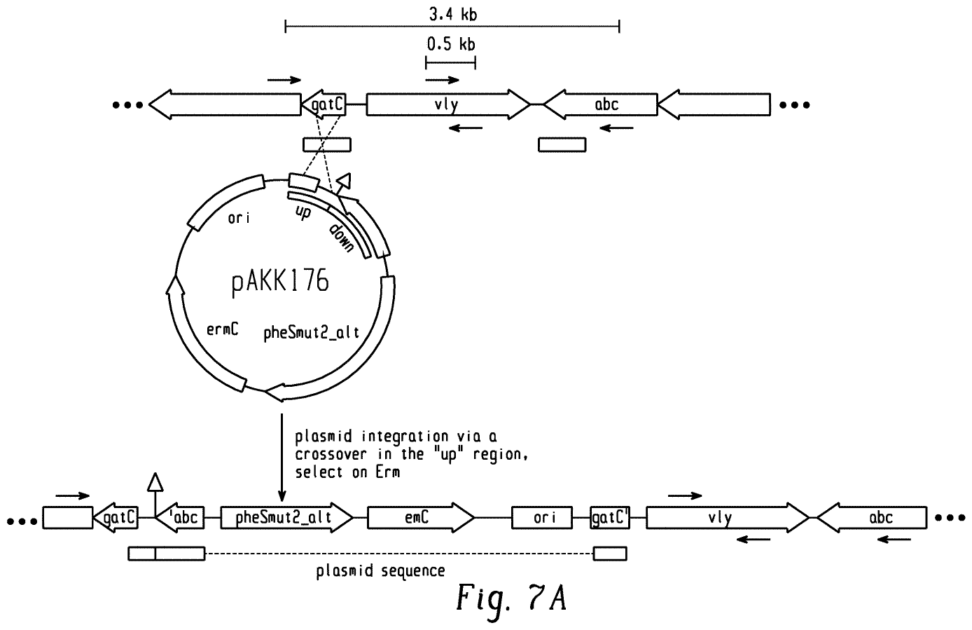

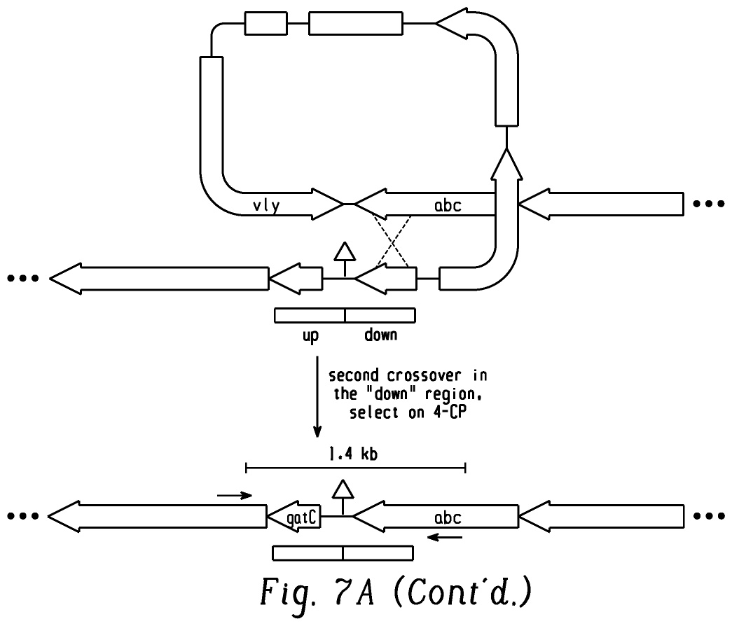

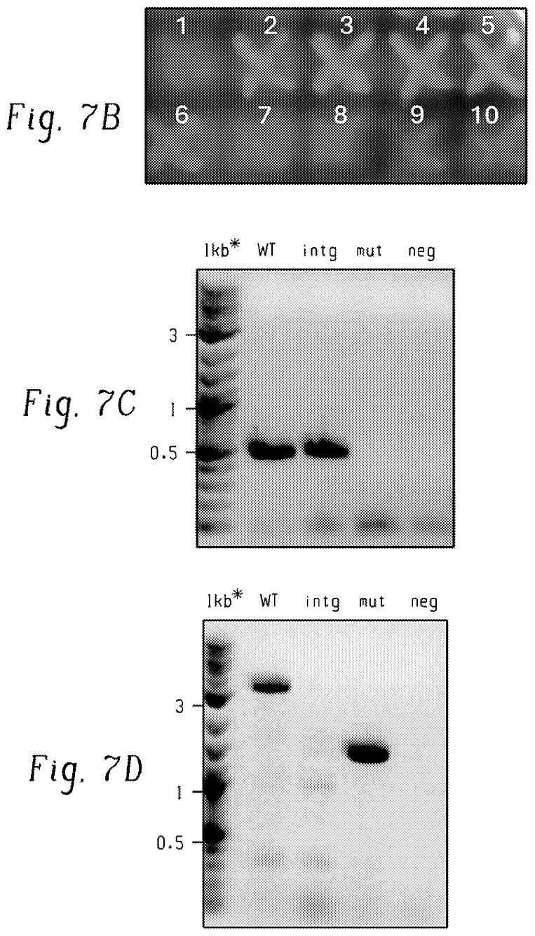

While working with derivatives of the positive/negative selection plasmid pAKK163, it was found that the plasmid sometimes recombined at pheS in the G. vaginalis chromosome instead of at our target gene. Therefore, to prevent recombination from occurring at pheS, the pheSₘᵤₜ₂ gene was recoded, substituting G. vaginalis codons with those optimized for Bifidobacterium longum, a related bacterial species. This process resulted in the positive/negative selection construct pAKK174. This new vector was used to delete the entire vaginolysin coding region. A plasmid that fused approximately 0.5 kb of DNA upstream and downstream of vly cloned in pAKK174 was designed, resulting in pAKK176 (FIG. 7A). Upon transformation of ATCC 14018, Erm^{R} transformants were obtained that had pAKK176 integrated in the region near vly at a frequency of 1.4×10⁻⁸, approximately 1 log higher than the frequency of spontaneous Erm^{R} colonies. After growing one of these isolates without Erm, the culture was plated on medium containing 4CP. Isolates that grew on 4CP and failed to grow on Erm-containing medium were patched to HBT to screen for those that had retained the vly deletion, evidenced by diminished hemolysis (FIG. 7B). PCR analysis of one of these non-hemolytic mutants confirmed it lacked vly (FIGS. 7C and 7D).

### Example 11: Complementation of the Vly Deletion Mutant

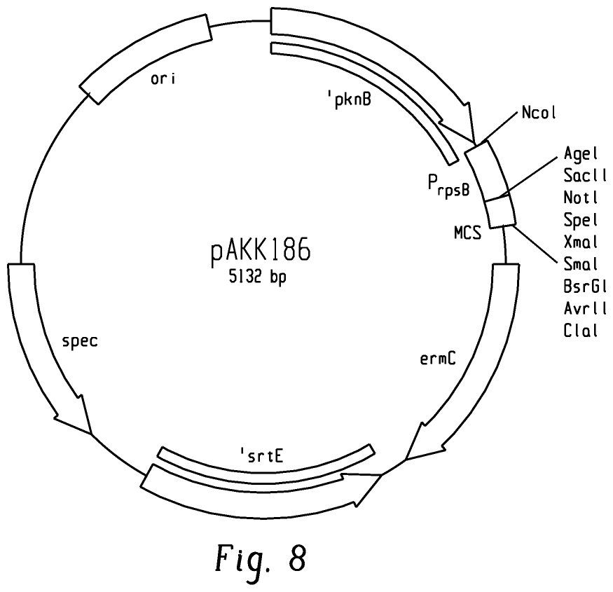

To identify a site in the G. vaginalis genome that could be used to express genes for complementation, the genome was scanned for a region that had a pair of convergent genes and a conserved chromosomal organization across different Gardnerella species. The region containing pknB, encoding a serine/threonine protein kinase, and srtE, coding for a class E housekeeping sortase, met these criteria. A construct was designed that contained approximately 0.8 kb of the 3′ end of pknB, and 0.8 kb of the 3′ end of srtE, along with a multiple cloning site (MCS) between these two gene fragments. To drive constitutive expression of the gene of interest, the promoter region from rpsB of ATCC 14018 was included. Restriction sites on either side of the rpsB promoter (NcoI/AgeI) allow for the possibility of cloning in a different promoter. The complementation construct, pAKK186, has a spectinomycin resistance gene on the vector and the ermC gene within the region that will be incorporated into the chromosome (FIG. 8).

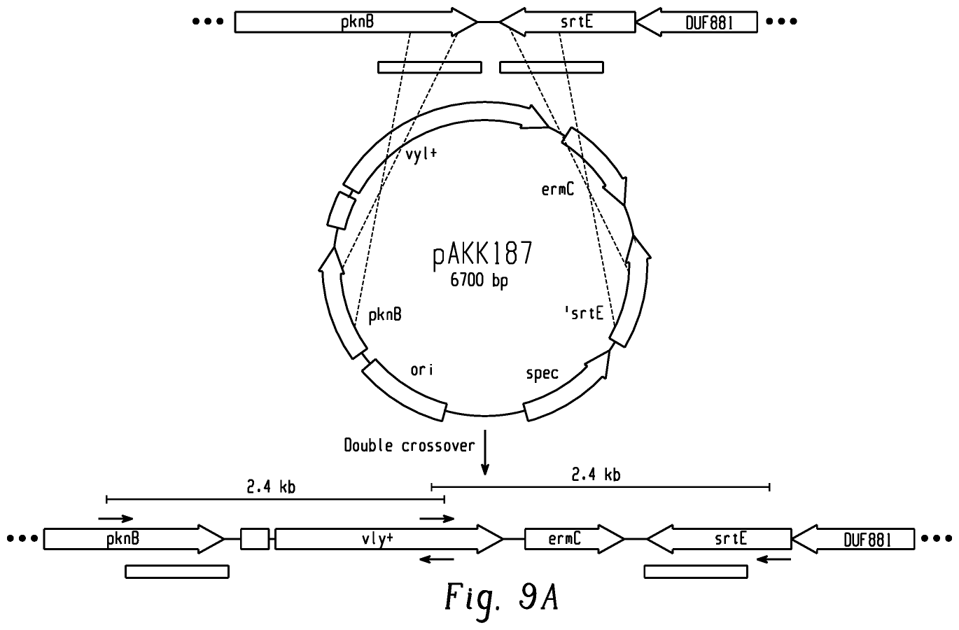

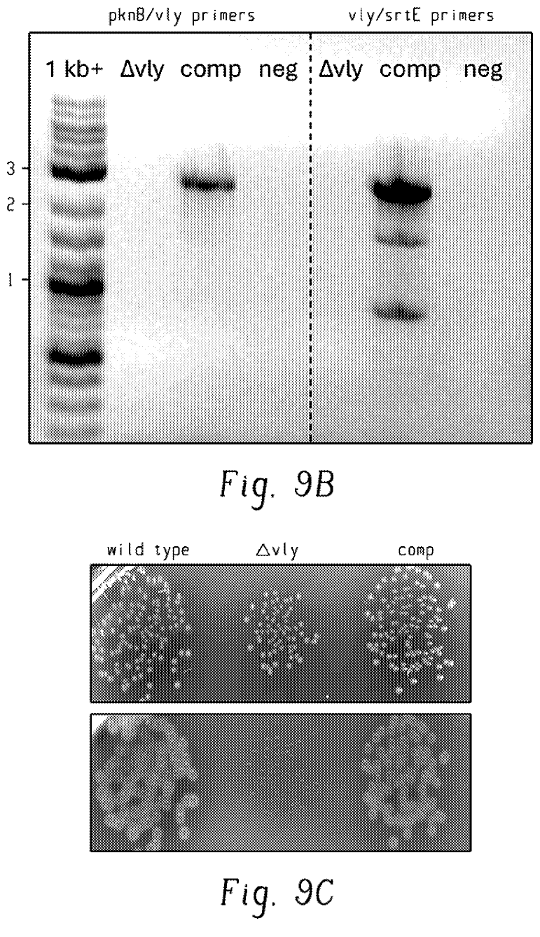

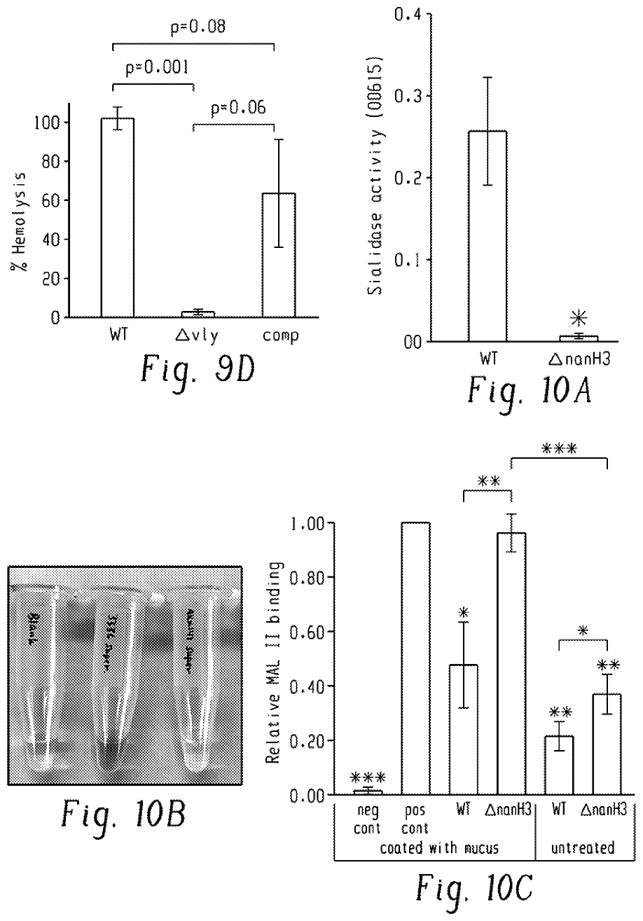

The wild-type vly gene was PCR-amplified, including its RBS, from ATCC 14018 and directionally cloned this sequence into pAKK186, resulting in pAKK187 (FIG. 9A). After treatment with HaeIII methyltransferase, the plasmid was transformed into the vly deletion mutant, AKK132. Five Erm^{R} transformants were patched to plates containing spectinomycin to determine whether the isolates had the plasmid integrated via a single crossover and would be Erm^{R} Spec^{R}, or had undergone double-crossover recombination, which would result in Erm^{R} only. Two of the five isolates were resistant to both antibiotics, while three were resistant to Erm only. One of the Erm^{R} Spc^{S} isolates was analyzed by PCR to confirm that it had undergone double-crossover recombination and incorporated vly at the pknB-srtE locus (FIG. 9B). Spotting dilutions of the wild type, vly deletion mutant (AKK132), and complemented mutant (AKK136) on HBT agar showed that the mutant is non-hemolytic, while the complement exhibited the hemolytic phenotype like the wild-type strain (FIG. 9C). In quantitative assays, the wild type was strongly hemolytic and the vly mutant showed virtually no hemolytic activity on human red blood cells. The complemented mutant appeared only somewhat reduced in hemolysis activity compared to the wild type and was statistically not different from wild type (FIG. 9D).

### Example 12: Mutagenesis in a Different Gardnerella Species, a Sialidase Deletion Mutant in G. pickettii

To determine if the methods described herein would work for other Gardnerella species, the sialidase gene nanH3 was deleted in G. pickettii strain 3336. Two regions of the 3336 chromosome that were upstream or downstream of the nanH3 gene PCR-amplified and were each approximately 0.8 kb. These DNA fragments were ligated into the positive/negative selection plasmid pAKK174 such that they created an in-frame deletion of nanH3, omitting 95% of the nanH3 coding sequence. The resulting plasmid, pAKK191, was transformed into 3336, and Erm^{R} transformants were obtained at a frequency of 1×10⁻⁶. After overnight growth of one of these transformants without antibiotic, plating of the bacteria on BHI-YDS agar containing 4-CP yielded isolates that had lost the plasmid insertion. PCR screening of seven isolates identified four that had lost the insertion and kept the nanH3 deletion, and DNA sequencing verified the deletion. The chromogenic substrate X-Neu5Ac was used to assay for sialidase activity in the Gardnerella strains. Lysates of wild-type 3336 exhibited significant sialidase activity whereas little or no sialidase activity was present in lysates from the nanH3 mutant. (FIGS. 10A and B) These results demonstrate that our mutagenesis methods and tools are not limited in their use to only G. vaginalis but also work for other Gardnerella species and generate mutations at a similar frequency.

### Discussion Examples 1-12

Described herein is a set of tools and methods for genetic manipulation of Gardnerella species. Getting the methods to work for transformation and to produce antibiotic resistant Gardnerella isolates at a frequency substantially above the spontaneous antibiotic resistance frequency required overcoming several hurdles. Growth of Gardnerella in the presence of DCS significantly increased transformation frequency, possibly due to affecting the barrier function of the envelope. DCS inhibits biosynthesis of both D-Ala and the D-Ala-D-Ala dipeptide used for making the UDP-MurNAc pentapeptide that is a precursor to peptidoglycan. The D-Ala-D-Ala portion of newly added peptidoglycan is used in the transpeptidation reaction for crosslinking peptidoglycan strands together. Thus, DCS-treated Gardnerella may have less cell wall or may have less-crosslinked cell wall. Another step that increased transformation frequency was the use of circular DNA. The reason for this difference is unclear, but it is possible that linear constructs were more subject to Gardnerella nucleases. Finally, a restriction barrier had to be overcome. Methylation of transforming DNA or knock-out of the haeIIIR gene encoding the HaeIII-like restriction enzyme in ATCC 14018 was effective in greatly increasing transformation. This last result is consistent with the results Kularatne and Hill observed when developing a method for transforming G. vaginalis with a Bifidobacterium-E. coli shuttle plasmid. Their shuttle plasmid was digested in vitro if treated with ATCC 14018 lysate but not with ATCC 49145 lysate, and they then used ATCC 49145 for their transformations (Kularatne, BMDN and Hill, J E. 2025. Transformation of Gardnerella vaginalis with a Bifidobacterium-Escherichia coli shuttle vector plasmid. BioRxiv 10.1101/2025.02.14.638355). Plasmids with larger regions of homology to the Gardnerella chromosome yielded increased frequencies for insertion mutants, as was previously noted in Neisseria gonorrhoeae and Streptococcus pneumonia (Hamilton, H L, Schwartz, K J, and Dillard, J P. 2001. Insertion-duplication mutagenesis of Neisseria: Use in characterization of DNA transfer genes in the gonococcal genetic island. J Bacteriol. 183: 4718-4726; Lee, M S, Seok, C, and Morrison, D A. 1998. Insertion-duplication mutagenesis in Streptococcus pneumoniae: targeting fragment length is a critical parameter in use as a random insertion tool. Appl Environ Microbiol. 64: 4796-802). Insert sizes of 500-1800 bp gave similar high transformation frequencies, but even a plasmid with an insert of 200 bp gave transformants. Overall, the methods described herein improved targeted mutagenesis in Gardnerella from undetectable above background spontaneous resistance at 10⁻⁹ antibiotic resistant CFU/total CFU to approximately 10⁻⁶ antibiotic resistant CFU/total CFU for the recombinant strains.

Insertion mutations, a point mutation, and a deletion were produced in the vaginolysin gene. Vaginolysin is a pore-forming toxin that lyses human red blood cells, epithelial cells, and neutrophils. Vaginolysin binds human CD59, and this specificity likely explains the human-specific nature of Gardnerella infection. The role of vaginolysin in infection is debated. It has been proposed to act in nutrient acquisition, adherence, detachment from the cell surface, or destruction of neutrophils to facilitate immune evasion. However, in a three-dimensional cell culture model, CD59 was found on the basolateral surface of cells, and vaginolysin added to the apical side failed to permeabilize the human cells. Others found that vaginolysin added to human vaginal or cervical cell lines resulted in membrane vesicle release from the human cells rather than lysis. In the in vitro assays, all of the vly mutants lost hemolytic activity for human red blood cells. To test vaginolysin function with a more relevant tissue, human cervix tissue was infected in organ culture. Lactate dehydrogenase assays demonstrated that infection with wild-type G. vaginalis resulted in cell lysis in the cervix tissue, and infection with the vaginolysin mutant resulted in levels of cell lysis no different than uninfected control.

A sialidase mutant of G. pickettii strain 3336 was also produced. Sialidase activity is only found in some Gardnerella clades and species. Sialidase is considered to be a likely virulence factor since sialic acid caps some glycan strands on mucins, preventing their degradation. Mutation of the nanH3 gene in 3336 was sufficient to eliminate sialidase activity as measured with a chromogenic substrate. Sialidase is thought to degrade mucus for nutrient acquisition or to allow Gardnerella to access the host cell surface. Gardnerella can also use sialidase to desialylate immunoglobulin A, facilitating its degradation and preventing it from functioning in immune clearance of the bacteria.

New treatments are needed for BV. Antibiotic treatment only clears the infection in 60-70% of patients. Furthermore, many patients that are successfully cured, relapse within a number of months. To better treat BV, we need to identify the essential virulence factors. Using our mutagenesis methods and tools, Gardnerella mutants can be made and tested for loss of virulence in human cell or tissue infection in vitro or in the estradiol-treated mouse model. Identified virulence factors would serve as targets for antibiotics or be used in vaccines. Deletion mutants lacking genes for virulence factors, such as vaginolysin or sialidase, might be able to colonize the vagina without causing disease. Thus, vaginal microbiota transplants containing such nonpathogenic bacteria might be used after antibiotic treatment to occupy the niche and prevent reinfection with BV-causing bacteria. Furthermore, loss of factors that Gardnerella use to fight the immune system (such as sialidase and vaginolysin) might allow the adaptive immune system to develop protective responses that would block or clear any Gardnerella infection and thus prevent BV.

### Example 13: HaeIIIR Deletion Mutant of G. vaginalis Strain 14018

To make a stable mutant of G. vaginalis 14018 that would not restrict transforming DNA, an in-frame deletion in the haeIIIR gene was made, removing 279 base pairs of the coding sequence. Synthetic DNA identical to 493 bp upstream of the desired deletion fused to 487 downstream of the desired deletion was made corresponding to those sequences in the G. vaginalis 14018 genome sequence. The constructed DNA was introduced into the positive/negative selection plasmid pAKK174, generating pAKK177. The plasmid pAKK177 was treated with HaeIII methylase and transformed into G. vaginalis strain 14018, generating strain AKK133. AKK133 was grown without selection to allow for plasmid resolution, and 4-CP selection yielded isolates that had lost the plasmid insertion but retained the haeIIIR deletion. One such isolate was designated AKK134. DNA sequencing confirmed that this strain carried the desired 279 bp deletion in the haeIIIR gene.

### Sequence of the Chromosome in Strain AKK134.

(SEQ ID NO: 37)
CATTGGACTGCAAACTGGAAGATATCATGGAAAACGTAGAAGAATAAGGA
GATTTTAAATGATTATTAGAACATATGGTTGGATTATTGAAAATCTGTAT
CGCTTTAAACTGCAATATTAGTGACATTATGGAAATACAAAAAGAAATGA
GGTAAAAAAAATTATGGCAAAAACATTTGGTTCCGAAGAACATAAAATAT
TATCACTGTTTAGCGCTGACTCAACATTTTGTTATGAAGGAGAATTCTTT
AAAGTAAATAATTCTGGAAAGCCAACCTGCAAAAAAGGTGAACCTAAAAC
TGATATATATGTAGAAGCTGTTAATAGTAACAACTGTGTTAAAGAGTTTA
AAATCTCTTTTAAGCAAGAAAATGCTGAATTTCTTGAAAATAAAACCAAT
GCTGAGCGAGCAGAACAGTTATTCGGATCTAACTGGCAAGAGATTATTAT
CAGCGCTGTGACTAAATTACAGAATGATTTTCAAAAAAGACCCATTGATG
ATTACGTTGATCAGAATCCAAATGTGTATTTTGCTTGTAAAGCGTTAAAT
TATCGTTCACTTAAAAATAAATATGATGGTGACAGACCTTTGGCTGTTTA
TGTAAATTGGTTCGTAACTAATGGAAAATTGGATTATGAATTAGTATTTG
ATAAACCCCTTTTGGAAGGGGGAGATGCTGTATATGAACGATTAAAATCA
GCTTTAGATTTATTAGGCATTTCCAATACTAATGATTTAAGTCTTGAATT
AATAATGCATACTGAATCTGTGAACATATAGGCATAGATGAGTGGTGGAA
GCTTTAAACTCAGCTCCCACCACTTATCTGAATTATGATAGAACTTTTTT
AATTGCAGTAGCTATTTCAAATGCTAGATTTACTGGTACTGCATTCCCAA
TCATTTTATAACCCGTATCTAAGCTATCGTATATGAACTTAAAATTATCT
GGAAATCCTTGTATTCTGGCTATTTCTC

### Sequence of the Plasmid Used to Make the Deletion.

pAKK177 (in-frame deletion of haeIIIR) whole
plasmid sequence
(SEQ ID NO: 38)
GCGGCCGTAAAACGACGGCCAGTGAGCGCGCGTAATACGACTCACTATAG
GGCGAATTTCTAGAGGAGCTCCACCGCGGTGGCGGCCGCACTAGTCATTG
GACTGCAAACTGGAAGATATCATGGAAAACGTAGAAGAATAAGGAGATTT
TAAATGATTATTAGAACATATGGTTGGATTATTGAAAATCTGTATCGCTT
TAAACTGCAATATTAGTGACATTATGGAAATACAAAAAGAAATGAGGTAA
AAAAAATTATGGCAAAAACATTTGGTTCCGAAGAACATAAAATATTATCA
CTGTTTAGCGCTGACTCAACATTTTGTTATGAAGGAGAATTCTTTAAAGT
AAATAATTCTGGAAAGCCAACCTGCAAAAAAGGTGAACCTAAAACTGATA
TATATGTAGAAGCTGTTAATAGTAACAACTGTGTTAAAGAGTTTAAAATC
TCTTTTAAGCAAGAAAATGCTGAATTTCTTGAAAATAAAACCAATGCTGA
GCGAGCAGAACAGTTATTCGGATCTAACTGGCAAGAGATTATTATCAGCG
CTGTGACTAAATTACAGAATGATTTTCAAAAAAGACCCATTGATGATTAC
GTTGATCAGAATCCAAATGTGTATTTTGCTTGTAAAGCGTTAAATTATCG
TTCACTTAAAAATAAATATGATGGTGACAGACCTTTGGCTGTTTATGTAA
ATTGGTTCGTAACTAATGGAAAATTGGATTATGAATTAGTATTTGATAAA
CCCCTTTTGGAAGGGGGAGATGCTGTATATGAACGATTAAAATCAGCTTT
AGATTTATTAGGCATTTCCAATACTAATGATTTAAGTCTTGAATTAATAA
TGCATACTGAATCTGTGAACATATAGGCATAGATGAGTGGTGGAAGCTTT
AAACTCAGCTCCCACCACTTATCTGAATTATGATAGAACTTTTTTAATTG
CAGTAGCTATTTCAAATGCTAGATTTACTGGTACTGCATTCCCAATCATT
TTATAACCCGTATCTAAGCTATCGTATATGAACTTAAAATTATCTGGAAA
TCCTTGTATTCTGGCTATTTCTCCCATCaaGcttatcgataCCGTCGACC
TCGAGCGTAATGCGTCGTACATACCGCACATTAAAATGAACATGTTAGTT
TAAATAACGAAGACCAAAACGTATTGTTAAAAATCGCGAAAAATCGCTAG
AACTATGCGTTAAGGTTTCACTCAAGGGAGATCCCATATGtTGCATAAAT
CtACGCACAACACtGACCGcAAAGGaGCCGTGGCGTCtAGCAAGCCGTTC
GACGCTAAAGCTATTACtAAAGTTGTGAAAGAAGGtATaGCCTGCGTaCA
GGCaGCTAAAACaATGGAAGAGtTGAAGGCGGCTAAGACGAAGTATGCCG
GCGCTCAATCCGCTATGACaCTtGCaAGCAAAAGCATTGGATCCtTGCAA
GTTGAGGAGAAGAAGGAAGCTGGTAAGATtATGTCtGCTtTGCGcGCGGA
TTTTGGCCGcGAGTTCGCTGCaGCCCAAGAGCGCATTAAAGCGATtGAAG
AGGCTAATATGCTtCAAAAGGAAACGGTaGATATGACatTGCCGATaAAC
CGCAAACCGCTtGGAGCaCGTCATCCGATTGCGCGTATTATTGAAGATTT
CGAGGACTTCTTCGTGTCtATGGGATGGCAAATTTCtGCGGGTCCGGAAG
TTGAGACGGAGTGGTTTGATTTCGACGCGTGAATTTCGGTCCaGACCACC
CaGCTCGTCAGATGCAGGATACCTTCTACGTaCAAGGAAATCAGGCCAAA
GATGCGGCTGGATTTGTTGGaTCGAATATGGTGCTtCGTACaCAGACGTC
tTCtGATCAAGTTCGTGCatTGATTGAACGTGGTGTTCCaCTtTATATTG
CGAGCCCGGGCAGGGTGTTCAGGACtGACGAACTtGACGCGACtCACACC
CCaGTGTTTCATCAATGCGAGGCGCTtGCTGTGGATAAGCATCTTCCATG
GCCGACCTtAAAGGCGTTCTtGATAGGCTtGCTGTTGCaATGTTCGGCCC
aGACGCGAAGTCtCGTCTGCGCCCGTCtTACTTTCCGTTTAGCGAACCGT
CtGCGGAAtTGGACCTtTGGTTTCCGGACAAAAAAGGAGGACCaGGCTGG
ATaGAATGGGGGGATGCGGCATGGTGAACCCaAATGTaCTtAAATCCGCT
GGTCTtGACCCaGACGTTTACACGGGaTTTGGTTTTGGtGTCGGTCTtGA
ACGcACaCTGtTGCTGAGGCATGACATtAACGATATGCATGACtTGGTaG
AGGGCGACAAACGTTTCTCtGAGCAGTTTGTTATGGGTGAGTAGGGTACC
GGGGCAGCTTTTGTTCCCTTTAGTGAGGGTTAATTGCGCGCTTGGCGTAA
TCATGGTCATAGCTGTTCGATAAGCTAATTCTCATGTTTGACAGCTTATC
ATCGCGTGCTATAATTATACTAATTTTATAAGGAGGAAAAAATAAAGAGG
GTTATAATGAACGAGAAAAATATAAAACACAGTCAAAACTTTATTACTTC
AAAACATAATATAGATAAAATAATGACAAATATAAGATTAAATGAACATG
ATAATATCTTTGAAATCGGCTCAGGAAAAGGGCATTTTACCCTTGAATTA
GTACAGAGGTGTAATTTCGTAACTGCCATTGAAATAGACCATAAATTATG
CAAAACTACAGAAAATAAACTTGTTGATCACGATAATTTCCAAGTTTTAA
ACAAGGATATATTGCAGTTTAAATTTCCTAAAAACCAATCCTATAAAATA
TTTGGTAATATACCTTATAACATAAGTACGGATATAATACGCAAAATTGT
TTTTGATAGTATAGCTGATGAGATTTATTTAATCGTGGAATACGGGTTTG
CTAAAAGATTATTAAATACAAAACGCTCATTGGCATTATTTTTAATGGCA
GAAGTTGATATTTCTATATTAAGTATGGTTCCAAGAGAATATTTTCATCC
TAAACCTAAAGTGAATAGCTCACTTATCAGATTAAATAGAAAAAAATCAA
GAATATCACACAAAGATAAACAGAAGTATAATTATTTCGTTATGAAATGG
GTTAACAAAGAATACAAGAAAATATTTACAAAAAATCAATTTAACAATTC
CTTAAAACATGCAGGAATTGACGATTTAAACAATATTAGCTTTGAACAAT
TCTTATCTCTTTTCAATAGCTATAAATTATTTAATAAGTAAGTTAAGGGA
TGCATAAACTGCATCCCTTAACTTGTTTTTCGTGTACCTATTTTTTGTGA
ATCGACTCTAGAGCTTGCCGTCTGAAATGGTTTCAGACGGCATGCAGCAA
TGGCAACAACGTTGAAGCTAGCTTATGCGGTGTGAAATACCGCACAGATG
CGTAAGGAGAAAATACCGCATCAGGCGCTCTTCCGCTTCCTCGCTCACTG
ACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAAT
CGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTC
CAAGCTGGGCTGTGTGCACGAACCCCCCGTTCAGCCCGACCGCTGCGCCT
TATCCGGTAACTATCGTCTTGAGTCCAACCCGGTAAGACACGACTTATCG
CCACTGGCAGCAGCCACTGGTAACAGGATTAGCAGAGCGAGGTATGTAGG
CGGTGCTACAGAGTTCTTGAAGTGGTGGCCTAACTACGGCTACACTAGAA
GGACAGTATTTGGTATCTGCGCTCTGCTGAAGCCAGTTACCTTCGGAAAA
AGAGTTGGTAGCTCTTGATCCGGCAAACAAACCACCGCTGGTAGCGGCGG
TTTTTTGTTTGCAAGCAGCAGATTACGCGCAGAAAAAAAGGATCTCAAGA
AGATCCTTTGATCTTTTCTTACTGAACGGTGATCCCCACCGGAATT

### Example 14: Mutant of G. vaginalis Strain 14018 Lacking a Putative Adhesin, the FIVAR Repeat Protein

G. vaginalis carries a gene for a putative adhesin which referred to herein as Frp1, for FIVAR repeat protein 1. The gene is designated E3D8E1 in the genome sequence. The Frp1 protein could aid in the attachment of the bacteria to human cells and tissue since FIVAR repeats in proteins in other bacteria have been shown to mediate attachment of the bacteria to fibronectin in host tissue (Christner, M., G. C. Franke, N. N. Schommer, U. Wendt, K. Wegert, P. Pehle, G. Kroll, C. Schulze, F. Buck, D. Mack, M. Aepfelbacher and H. Rohde (2010). “The giant extracellular matrix-binding protein of Staphylococcus epidermidis mediates biofilm accumulation and attachment to fibronectin.” Mol Microbiol 75(1): 187-207). A G. vaginalis mutant carrying a mutation in frp1 would be useful for determining the factors necessary for the bacteria to adhere to host tissue and could be used to make strains that could serve in vaccine formulations or as probiotics.

Synthetic DNA 0.5 kilobases in length was constructed that was identical to a portion of the frp1 gene in G. vaginalis 14018 except for the introduction of a stop codon within the coding sequence, changing a GAA codon sequence to TAA. The synthetic DNA was introduced into plasmid pAKK174, resulting in plasmid pAKK198. Plasmid pAKK198 was transformed into G. vaginalis 14018 haeIIIR mutant AKK134, resulting in strain ZMS100. Following growth without selection, 4-CP resistant isolates were obtained that lost the plasmid insertion and retained the mutation. One such isolate was chosen, designated ZMS101, and the DNA sequence confirmed that the strain carries the mutation in frp1.

ZMS101 Chromosome Sequence with the Location of the Mutated Sequence Underlined, and the Single Mutated Base in Bold. (SEQ ID NO: 39)

GAATAAGACTGAGGGTGAGGATTCTAAGAAGACTGCGACCGAGACTAATA
AGAAGGAAGAATCTAAGAAGGAGCAGCCTGAGCAGACTAAACCAACTGAG
CAGACTGTAGTAAAAACCGAAGGTAAGGCTGACGCTACTAAGACTGAGTC
TGGGTCGAAGGTTGAGAGCGCTACGGCTGTAGATAATAAGGTTACGAATA
GCACTTCTGCTACTGATAAGAATAATGCAGACAGTAGCACCACTGAGGAA
ACTAGCACTCCTGCGGAAAATGTTGTAAAGAAGCGTACAAGGCGTGCGAC
GGTTGAGAATAAGCAACCTGAGGCTACTAATTATGCTAAGGATGAAAATC
AGAAGCAGAGCGAACCTGTTGTTGGTCAAGATCGCGAAGCTAATCCTGCA
CCTGCTCCACAAGTTAATAGTGAGATAACGCTTTAAAAAGAAACAAAAGA
CACGCTTCCTAACTTGTATGCATGGGGATCTTCTGACAATGTGTATATTG
AAAAAGGGCAAAACCAAGAAGTAACATTTAATTTTGCAAAACCTTCTGAC
GGTAGCACAATTACCAAAGTAGCAATTTTCCCATCTGATGGTAATACTGT
TGATAATGATAAAAGTAGAAAGTTCCTTGAATATTATTCTGCTAATGAAA
ACGAGCACAAGCCATATTC

### Plasmid Used to Make the Frp1 Stop Mutation.

pAKK198 FIVAR stop whole plasmid sequence
(SEQ ID NO: 40)
GCGGCCGTAAAACGACGGCCAGTGAGCGCGCGTAATACGACTCACTATAG
GGCGAATTTCTAGAGGAGCTCCACCGCGGTGGCGGCCGCACTAGTTAATA
AGGTTACGAATAGCACTTCTGCTACTGATAAGAATAATGCAGACAGTAGC
ACCACTGAGGAAACTAGCACTCCTGCGGAAAATGTTGTAAAGAAGCGTAC
AAGGCGTGCGACGGTTGAGAATAAGCAACCTGAGGCTACTAATTATGCTA
AGGATGAAAATCAGAAGCAGAGCGAACCTGTTGTTGGTCAAGATCGCGAA
GCTAATCCTGCACCTGCTCCACAAGTTAATAGTGAGATAACGCTTTCAAA
AGAAACAAAAGACACGCTTCCTAACTTGTATGCATGGGGATCTTCTGACA
ATGTGTATATTGAAAAAGGGCAAAACCAAGAAGTAACATTTAATTTTGCA
AAACCTTCTGACGGTAGCACAATTACCAAAGTAGCAATTTTCCCATCTGA
TGGTAATACTGTTGATAATGATAAAAGTAGAAAGTTCCTTGAATATTATT
CTGCTAATGAAAACGAGCACAAGCCATATTCTGGTAAGTACGAATTTgaa
ttcGaTATCaaGcttatcgataCCGTCGACCTCGAGCGTAATGCGTCGTA
CATACCGCACATTAAAATGAACATGTTAGTTTAAATAACGAAGACCAAAA
CGTATTGTTAAAAATCGCGAAAAATCGCTAGAACTATGCGTTAAGGTTTC
ACTCAAGGGAGATCCCATATGtTGCATAAATCtACGCACAACACtGACCG
cAAAGGaGCCGTGGCGTCtAGCAAGCCGTTCGACGCTAAAGCTATTACtA
AAGTTGTGAAAGAAGGtATaGCCTGCGTaCAGGCaGCTAAAACaATGGAA
GAGTGAAGGCGGCTAAGACGAAGTATGCCGGCGCTCAATCCGCTATGACa
CTtGCaAGCAAAAGCATTGGATCCTGCAAGTTGAGGAGAAGAAGGAAGCT
GGTAAGATtATGTCtGCTtTGCGcGCGGATTTTGGCCGcGAGTTCGCTGC
aGCCCAAGAGCGCATTAAAGCGATtGAAGAGGCTAATATGCTtCAAAAGG
AAACGGTaGATATGACatTGCCGATaAACCGCAAACCGCTtGGAGCaCGT
CATCCGATTGCGCGTATTATTGAAGATTTCGAGGACTTCTTCGTGTCtAT
GGGATGGCAAATTTCtGCGGGTCCGGAAGTTGAGACGGAGTGGTTTGATT
TCGACGCGtTGAATTTCGGTCCaGACCACCCaGCTCGTCAGATGCAGGAT
ACCTTCTACGTaCAAGGAAATCAGGCCAAAGATGCGGCTGGATTTGTTGG
aTCGAATATGGTGCTtCGTACaCAGACGTCtTCtGATCAAGTTCGTGCat
TGATTGAACGTGGTGTTCCaCTtTATATTGCGAGCCCGGGCAGGGTGTTC
AGGACtGACGAACTtGACGCGACtCACACCCCaGTGTTTCATCAATGCGA
GGCGCTtGCTGTGGATAAGCATCTtTCCATGGCCGACCTtAAAGGCGTTC
TtGATAGGCTtGCTGTTGCaATGTTCGGCCCaGACGCGAAGTCtCGTCTG
CGCCCGTCtTACTTTCCGTTTAGCGAACCGTCtGCGGAAtTGGACCTtTG
GTTTCCGGACAAAAAAGGAGGACCaGGCTGGATaGAATGGGGGGATGCGG
CATGGTGAACCCaAATGTaCTtAAATCCGCTGGTCTtGACCCaGACGTTT
ACACGGGaTTTGGTTTTGGtGTCGGTCTtGAACGcACaCTGtTGCTGAGG
CATGACATtAACGATATGCATGACtTGGTaGAGGGCGACAAACGTTTCTC
tGAGCAGTTTGTTATGGGTGAGTAGGGTACCtGGGGCAGCTTTTGTTCCC
TTTAGTGAGGGTTAATTGCGCGCTTGGCGTAATCATGGTCATAGCTGTTC
GATAAGCTAATTCTCATGTTTGACAGCTTATCATCGCGTGCTATAATTAT
ACTAATTTTATAAGGAGGAAAAAATAAAGAGGGTTATAATGAACGAGAAA
AATATAAAACACAGTCAAAACTTTATTACTTCAAAACATAATATAGATAA
AATAATGACAAATATAAGATTAAATGAACATGATAATATCTTTGAAATCG
GCTCAGGAAAAGGGCATTTTACCCTTGAATTAGTACAGAGGTGTAATTTC
GTAACTGCCATTGAAATAGACCATAAATTATGCAAAACTACAGAAAATAA
ACTTGTTGATCACGATAATTTCCAAGTTTTAAACAAGGATATATTGCAGT
TTAAATTTCCTAAAAACCAATCCTATAAAATATTTGGTAATATACCTTAT
AACATAAGTACGGATATAATACGCAAAATTGTTTTTGATAGTATAGCTGA
TGAGATTTATTTAATCGTGGAATACGGGTTTGCTAAAAGATTATTAAATA
CAAAACGCTCATTGGCATTATTTTTAATGGCAGAAGTTGATATTTCTATA
TTAAGTATGGTTCCAAGAGAATATTTTCATCCTAAACCTAAAGTGAATAG
CTCACTTATCAGATTAAATAGAAAAAAATCAAGAATATCACACAAAGATA
AACAGAAGTATAATTATTTCGTTATGAAATGGGTTAACAAAGAATACAAG
AAAATATTTACAAAAAATCAATTTAACAATTCCTTAAAACATGCAGGAAT
TGACGATTTAAACAATATTAGCTTTGAACAATTCTTATCTCTTTTCAATA
GCTATAAATTATTTAATAAGTAAGTTAAGGGATGCATAAACTGCATCCCT
TAACTTGTTTTTCGTGTACCTATTTTTTGTGAATCGACTCTAGAGCTTGC
CGTCTGAAATGGTTTCAGACGGCATGCAGCAATGGCAACAACGTTGAAGC
TAGCTTATGCGGTGTGAAATACCGCACAGATGCGTAAGGAGAAAATACCG
CATCAGGCGCTCTTCCGCTTCCTCGCTCACTGACTCGCTGCGCTCGGTCG
TTCGGCTGCGGCGAGCGGTATCAGCTCACTCAAAGGCGGTAATACGGTTA
TCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGG
TGGCGAAACCCGACAGGACTATAAAGATACCAGGCGTTTCCCCCTGGAAG
CTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTC
TTGAGTCCAACCCGGTAAGACACGACTTATCGCCACTGGCAGCAGCCACT
GGTAACAGGATTAGCAGAGCGAGGTATGTAGGCGGTGCTACAGAGTTCTT
GAAGTGGTGGCCTAACTACGGCTACACTAGAAGGACAGTATTTGGTATCT
GCGCTCTGCTGAAGCCAGTTACCTTCGGAAAAAGAGTTGGTAGCTCTTGA
TCCGGCAAACAAACCACCGCTGGTAGCGGCGGTTTTTTGTTTGCAAGCAG
CAGATTACGCGCAGAAAAAAAGGATCTCAAGAAGATCCTTTGATCTTTTC
TTACTGAACGGTGATCCCCACCGGAATT

### Example 14: Mutant of G. vaginalis Strain 14018 Lacking a Putative Adhesin, a Pilus

G. vaginalis 14018 encodes a putative pilin which we designated pil2, genome designation E2D727. The pilin is likely part of a pilus apparatus on the G. vaginalis surface and could serve to adhere the bacteria to host cells and tissues. Synthetic DNA containing 507 bases upstream of the pil2 gene fused to 564 bases of DNA downstream of pil2 was constructed, carrying a 6 base linker ACTAGT in between the two sequences. This synthetic DNA was introduced into plasmid pAKK174 to generate pAKK206. Plasmid pAKK206 was transformed into G. vaginalis AKK134 to generate AKK149. AKK149 was grown without selection, and 4-CP selection resulted in the final mutant AKK150. The pil2 mutation in AKK150 was confirmed by DNA sequencing.

Below is the sequence of the mutated region in the AKK150 chromosome. The sequence actagt in the mutant replaces 1657 bases that contained the pi/2 coding region in the Gardnerella vaginalis strain 14018 chromosome (GenBank accession number AP012332.1 for the wild type strain). (SEQ ID NO: 41)

GAAGATTGCGTGAACTGGAACATGCTGCTGAGCGTAGTGTGCGTGATTGG
CACGGGCACCATGGCGGCTGGGTTCTTCCTTGATCAGACCAAGTGGGGGC
GCGCGATGCTTGATGCGTTGTTGCGTAAGACACTGATTAAAGATTTTATC
TGCAAAACTGCCAAAAAGCTTCGCGCTTTAAGGCGACGTAGCGAAAGGTG
GCGATGCTGATGAAAAAATTTGCACGCAATCCTGCTAGTCGGCTAGTCGA
AAGAGTAGCGCTGGCTTGCAAGCTTGCGGTGCGATTCTCGCTAAAACTTG
TTGCGTACTACGATTCGCGTAAACATTAGCGCGATGAATAATTGTGGCTC
GTTCGCAAATTGTGGATTGATCACGAACCGTGGCTTGCTTACAGATTGTG
GCTTGCCTATATATCGCAACACAAAATAAACAATAAACATTAAACAATAA
CCGTATTTTCCTCAAAAACCTGCCCAAAAGGGCACTGATATAAAGGAGAA
GAAAGTGactagtTGATTTGCTCAGTTTGATTAGTAAATCATAAACGATT
GAGTCTAGGGCAAAAACCCTAGACTCAATCCTATGTGGACTGTTAAAGCA
ACAAAACCCGTCGTTTTCCGCTTTTTGTTGTTGATTTTGTGGTTTTAAGC
AACATTTTCGGTAAGTTTTTCCCGATTTTGTTGTTAATTTTGCCGTTTTG
AGCAACAAAAAGCGCAAAATCCCACTGCGTTTGTAGCAAAATCAAACCAA
ATATTATCTTTAATTCAATAGCTTGAGCTTGGAGGAGTTATGAAGCTAGC
CGATATTAGCAATTTGTGCCACTCGTTGAGTGAGCGCGCTGTTGCTAAAT
TGTTTAAGAATAAGGCGAAGCGTACCTCCAATGTGGCTGGCACGAGCAGT
GTGGCTGGTGCGAGTGGTTTGCTAGCGGATAAGTCGCAAGCAACCGAAGA
GCAAATTCCACGATCCAACTCAAGCAAAAATATAAACCAAAATACACTTA
AAACTAAAAAAATAACTAAAAAGAAAAGCACACTGATGCGTATCTTAGAG
CCGATTGCGCTGGTTCTTGCAGGCATT

Below is the sequence of the plasmid used to make the pil2 mutation.

pAKK206 (pil2 mutation construct) whole sequence
(SEQ ID NO: 42)
GCTTCAAAAGGAAACGGTAGATATGACATTGCCGATAAACCGCAAACCGC
TTGGAGCACGTCATCCGATTGCGCGTATTATTGAAGATTTCGAGGACTTC
TTCGTGTCTATGGGATGGCAAATTTCTGCGGGTCCGGAAGTTGAGACGGA
GTGGTTTGATTTCGACGCGTTGAATTTCGGTCCAGACCACCCAGCTCGTC
AGATGCAGGATACCTTCTACGTACAAGGAAATCAGGCCAAAGATGCGGCT
GGATTTGTTGGATCGAATATGGTGCTTCGTACACAGACGTCTTCTGATCA
AGTTCGTGCATTGATTGAACGTGGTGTTCCACTTTATATTGCGAGCCCGG
GCAGGGTGTTCAGGACTGACGAACTTGACGCGACTCACACCCCAGTGTTT
CATCAATGCGAGGCGCTTGCTGTGGATAAGCATCTTTCCATGGCCGACCT
TAAAGGCGTTCTTGATAGGCTTGCTGTTGCAATGTTCGGCCCAGACGCGA
AGTCTCGTCTGCGCCCGTCTTACTTTCCGTTTAGCGAACCGTCTGCGGAA
TTGGACCTTTGGTTTCCGGACAAAAAAGGAGGACCAGGCTGGATAGAATG
GGGTGGATGCGGCATGGTGAACCCAAATGTACTTAAATCCGCTGGTCTTG
ACCCAGACGTTTACACGGGATTTGGTTTTGGTGTCGGTCTTGAACGCACA
CTGTTGCTGAGGCATGACATTAACGATATGCATGACTTGGTAGAGGGCGA
CAAACGTTTCTCTGAGCAGTTTGTTATGGGTGAGTAGGGTACCCAGCTTT
TGTTCCCTTTAGTGAGGGTTAATTGCGCGCTTGGCGTAATCATGGTCATA
GCTGTTCGATAAGCTAATTCTCATGTTTGACAGCTTATCATCGCGTGCTA
TAATTATACTAATTTTATAAGGAGGAAAAAATAAAGAGGGTTATAATGAA
CGAGAAAAATATAAAACACAGTCAAAACTTTATTACTTCAAAACATAATA
TAGATAAAATAATGACAAATATAAGATTAAATGAACATGATAATATCTTT
GAAATCGGCTCAGGAAAAGGGCATTTTACCCTTGAATTAGTACAGAGGTG
TAATTTCGTAACTGCCATTGAAATAGACCATAAATTATGCAAAACTACAG
AAAATAAACTTGTTGATCACGATAATTTCCAAGTTTTAAACAAGGATATA
TTGCAGTTTAAATTTCCTAAAAACCAATCCTATAAAATATTTGGTAATAT
ACCTTATAACATAAGTACGGATATAATACGCAAAATTGTTTTTGATAGTA
TAGCTGATGAGATTTATTTAATCGTGGAATACGGGTTTGCTAAAAGATTA
TTAAATACAAAACGCTCATTGGCATTATTTTTAATGGCAGAAGTTGATAT
TTCTATATTAAGTATGGTTCCAAGAGAATATTTTCATCCTAAACCTAAAG
TGAATAGCTCACTTATCAGATTAAATAGAAAAAAATCAAGAATATCACAC
AAAGATAAACAGAAGTATAATTATTTCGTTATGAAATGGGTTAACAAAGA
ATACAAGAAAATATTTACAAAAAATCAATTTAACAATTCCTTAAAACATG
CAGGAATTGACGATTTAAACAATATTAGCTTTGAACAATTCTTATCTCTT
TTCAATAGCTATAAATTATTTAATAAGTAAGTTAAGGGATGCATAAACTG
CATCCCTTAACTTGTTTTTCGTGTACCTATTTTTTGTGAATCGACTCTAG
AGCTTGCCGTCTGAAATGGTTTCAGACGGCATGCAGCAATGGCAACAACG
TTGAAGCTAGCTTATGCGGTGTGAAATACCGCACAGATGCGTAAGGAGAA
AATACCGCATCAGGCGCTCTTCCGCTTCCTCGCTCACTGACTCGCTGCGC
TCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAA
AAGGCCAGCAAAAGGCCAGGAACCGTAAAAAGGCCGCGTCGCTGGCGTTT
TTCCATAGGCTCCGCCCCCCTGACGAGCATCACAAAAATCGACGCTCAAG
TCAGAGGTGGCGAAACCTGACAGGACTATAAAGATACCAGGCGTTTCCCC
CTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAAC
TATCGTCTTGAGTCCAACCCGGTAAGACACGACTTATCGCCACTGGCAGC
AGCCACTGGTAACAGGATTAGCAGAGCGAGGTATGTAGGCGGTGCTACAG
AGTTCTTGAAGTGGTGGCCTAACTACGGCTACACTAGAAGGACAGTATTT
GGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTG
ATCTTTTCTACTGAACGGTGATCCCCACCGGAATTGCGGCCGTAAACGAC
GGCCAGTGAGCGCGCGTAATACGACTCACTATAGGGCGAATTGGAGCTCG
AAGATTGCGTGAACTGGAACATGCTGCTGAGCGTAGTGTGCGTGATTGGC
ACGGGCACCATGGCGGCTGGGTTCTTCCTTGATCAGACCAAGTGGGGGCG
CGCGATGCTTGATGCGTTGTTGCGTAAGACACTGATTAAAGATTTTATCT
GCAAAACTGCCAAAAAGCTTCGCGCTTTAAGGCGACGTAGCGAAAGGTGG
CGATGCTGATGAAAAAATTTGCACGCAATCCTGCTAGTCGGCTAGTCGAA
AGAGTAGCGCTGGCTTGCAAGCTTGCGGTGCGATTCTCGCTAAAACTTGT
TGCGTACTACGATTCGCGTAAACATTAGCGCGATGAATAATTGTGGCTCG
TTCGCAAATTGTGGATTGATCACGAACCGTGGCTTGCTTACAGATTGTGG
CTTGCCTATATATCGCAACACAAAATAAACAATAAACATTAAACAATAAC
CGTATTTTCCTCAAAAACCTGCCCAAAAGGGCACTGATATAAAGGAGAAG
AAAGTGACTAGTTGATTTGCTCAGTTTGATTAGTAAATCATAAACGATTG
AGTCTAGGGCAAAAACCCTAGACTCAATCCTATGTGGACTGTTAAAGCAA
CAAAACCCGTCGTTTTCCGCTTTTTGTTGTTGATTTTGTGGTTTTAAGCA
ACATTTTCGGTAAGTTTTTCCCGATTTTGTTGTTAATTTTGCCGTTTTGA
GCAACAAAAAGCGCAAAATCCCACTGCGTTTGTAGCAAAATCAAACCAAA
TATTATCTTTAATTCAATAGCTTGAGCTTGGAGGAGTTATGAAGCTAGCC
GATATTAGCAATTTGTGCCACTCGTTGAGTGAGCGCGCTGTTGCTAAATT
GTTTAAGAATAAGGCGAAGCGTACCTCCAATGTGGCTGGCACGAGCAGTG
TGGCTGGTGCGAGTGGTTTGCTAGCGGATAAGTCGCAAGCAACCGAAGAG
CAAATTCCACGATCCAACTCAAGCAAAAATATAAACCAAAATACACTTAA
AACTAAAAAAATAACTAAAAAGAAAAGCACACTGATGCGTATCTTAGAGC
CGATTGCGCTGGTTCTTGCAGGCATTCTCGAGCGTAATGCGTCGTACATA
CCGCACATTAAAATGAACATGTTAGTTTAAATAACGAAGACCAAAACGTA
TTGTTAAAAATCGCGAAAAATCGCTAGAACTATGCGTTAAGGTTTCACTC
AAGGGAGATCCCATATGTTGCATAAATCTACGCACAACACTGACCGCAAA
GGAGCCGTGGCGTCTAGCAAGCCGTTCGACGCTAAAGCTATTACTAAAGT
TGTGAAAGAAGGTATAGCCTGCGTACAGGCAGCTAAAACAATGGAAGAGT
TGAAGGCGGCTAAGACGAAGTATGCCGGCGCTCAATCCGCTATGACACTT
GCAAGCAAAAGCATTGGATCCTTGCAAGTTGAGGAGAAGAAGGAAGCTGG
TAAGATTATGTCTGCTTTGCGCGCGGATTTTGGCCGCGAGTTCGCTGCAG
CCCAAGAGCGCATTAAAGCGATTGAAGAGGCTAATAT

### Example 15: Cervical Mucus Degradation Assay with the G. pickettii 3336 Mutant Lacking the Sialidase (AKK141)

The function of NanH3 was examined by testing its ability to degrade human cervical mucus. Mucus was collected from human cervix tissue grown in organ culture. Cultures of wild type strain 3336 or the nanH3 mutant were added to wells of a 96-well plate coated with cervical mucus and incubated for 24 hours to allow for mucus degradation. MALII lectin was added to bind remaining sialic acid residues in the cervical mucus, and the amount of MALII binding was measured. The wild type strain degraded more than 50% of the mucus while the nanH3 mutant showed no significant reduction in sialic acid-containing mucus (FIG. 10C). Interestingly, controls containing 3336 or the nanH3 mutant but without cervical mucus addition showed some lectin binding. This result indicates that either the bacteria have a sialic acid-containing molecule on their surface even in the absence of mucus or that the bacteria are able to bind the lectin directly. Overall, these results demonstrate that NanH3 acts in the degradation of human cervical mucus.

### Example 16: Assessment of Gardnerella Sialidase Activity on Cervical Mucus

Mucus was harvested from cervical tissue obtained from NDRI and stored at −20 °C. An aliquot of cervical mucus was mixed with an equal volume of 0.1% DTT in PBS and placed on a shaker for 1 hr at room temperature. A Bradford assay was conducted to measure the protein concentration of the suspension. The suspension was adjusted to ~150 μg/ml with PBS and 100 μl was applied to wells of a 96-well tissue culture plate. The plate was incubated at 4 °C overnight. 3336 and AKK141 (ΔnanH3) were grown overnight on HBT, then swabbed into NYCIII media lacking glucose and adjusted to an OD₆₀₀ of 0.2. Excess mucus was removed, and the wells were washed once with PBST (PBS+0.05% Tween® 20). 100 μl of the bacterial suspensions or media alone were added to the wells and the plate was incubated at 37 °C with 5% CO₂ for ~24 hr. The following day bacteria or media were aspirated, and the wells were washed three times with 200 μl of PBST. 100 μl of a 0.5 μg/ml solution of biotinylated Maackia amurensis Lectin II (MAL II) (Vector Laboratories) in PBS were applied to the wells and incubated at room temperature with gentle rocking for 1 hr. The wells were washed three times with PBST. 100 μl of HRP-conjugated streptavidin (R&D Systems), diluted 1:200 in PBS, were applied the wells and incubated at room temperature for 1 hr with rocking. Wells were washed three times with 200 μl of PBST, then 100 μl of TMB substrate (3,3′,5,5′ tetramethylbenzidine) were added to the wells and incubated 5 min at room temperature. 50 μl of 2N sulfuric acid were added to stop the reaction and the color was quantified by measuring the OD₄₅₀ on a BioTek Synergy™ HT plate reader. Controls included wells that were pre-coated with cervical mucus but not treated with bacteria; the positive control had MAL II applied, while the negative control had PBS applied instead. Additional controls included bacterial suspensions that were added to wells that had not been pre-coated with cervical mucus.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A genetically modified Gardnerella strain, wherein the genetically modified Gardnerella strain has an insertion, a point mutation, a deletion, or a combination thereof, in a gene encoding a virulence factor, an adhesin, or a biofilm factor.

2. The genetically modified Gardnerella strain of claim 1, wherein the virulence factor is a vaginolysin, a sialidase, a phospholipase C, a prolidase, a glycosulfatase, a Tad pili, a sortase-dependent pili, a fibrontectin-binding protein, a collagen-binding protein, an albumin-binding protein, a beta-galactosidase, a fucosidase, or a combination thereof.

3. The genetically modified Gardnerella strain of claim 1, comprising G. vaginalis, G. picketti, G. piotii, G. leopoldii, G. swidsinskii, or G. greenwoodii.

4. The genetically modified Gardnerella strain of claim 1, wherein the strain is a G. vaginalis strain comprising an insertion of an antibiotic resistance gene, a nonsense mutation at E155 (GAA to TAA), or a deletion of all or a portion of the vaginolysin gene coding region.

5. The genetically modified Gardnerella strain of claim 1, wherein the strain is a G. pickettii strain comprising a deletion of all or a portion of a nanH3 sialidase gene, a G. vaginalis strain 14018 with a haeIIIR deletion, a G. vaginalis strain 14018 with a FIVAR repeat protein 1 (E3D8E1) deletion, a G. vaginalis strain 14018 with a pilus pil2 (E2D727) deletion, or a G. pickettii strain with a sialidase (nanH3) deletion.

6. A pharmaceutical composition comprising one or more genetically modified Gardnerella strains according to claim 1, and a pharmaceutically acceptable excipient.

7. The pharmaceutical composition of claim 6, in the form of an oral dosage form or a vaginal dosage form.

8. The pharmaceutical composition of claim 6, further comprising an additional bacteria selected from Bifdobacteria, Lactobacillus acidophilus, Lactobacillus rhamnosus, Lactobacillus reutri, Lactobacillus fermentum, Lactobacillus paracasei, Lactobacillus brevis, Lactobacillus salivarius, Lactobacillus plantarum, Lactobacillus crispatus, Lactobacillus iners, Lactobacillus jensenii, Lactobacillus gasseri, Lactobacillus casei, and combinations thereof.

9. The pharmaceutical composition of claim 6, further comprising boric acid, a prebiotic, a buffering agent, or a combination thereof.

10. The pharmaceutical composition of claim 9, wherein the prebiotic comprises sucrose, inulin, a fructooligosaccharide, a galactooligosaccharide, a hemicellulose, chitin, lactulose, a mannan oligosaccharide, oligofructose-enriched inulin, a gums, oligofructose, oligodextrose, tagatose, a resistant maltodextrin, a trans-galactooligosaccharide, a pectin, a dietary fiber, an xylooligosaccharide, or a combination thereof.

11. The pharmaceutical composition of claim 6 in the form of a vaccine composition.

12. A method of treating a woman in need of treatment for a disease associated with an imbalance of female reproductive system microbiota, comprising,
administering the pharmaceutical composition of claim 6 to the woman.

13. The method of claim 12, wherein the disease associated with an imbalance of female reproductive system microbiota comprises bacterial vaginosis, pelvic inflammatory disease, sexually transmitted infection, postpartum endometritis, acute endometritis, chronic endometritis, postpartum sepsis, likelihood of preterm birth, likelihood of preterm premature rupture of membranes, likelihood of miscarriage, miscarriage, recurrent miscarriage, chorioamnionitis, intra-amniotic infection, infertility, sterility, likelihood of ineffectiveness of fertility treatment, implantation failure, ectopic pregnancy, placenta previa, placental abruption, intrauterine growth restriction, pre-term delivery, preeclampsia, premature membrane rupture cervical intraepithelial neoplasia, cervical cancer, other cancers of the female genitourinary system, decreasing ammonia in the vaginal environment, reducing inflammation, preventing overgrowth of at least one of Gardnerella vaginalis and Prevotella spp. in the vagina, or a combination thereof.

14. The method of claim 12, wherein the woman has bacterial vaginosis or is at risk of developing bacterial vaginosis.

15. A method of preparing a genetically modified Gardnerella strain, comprising culturing Gardnerella cells in the presence of D-cycloserine (DCS);
transforming the cells with a polynucleotide comprising a target modification;
plating the cells; and
isolating the genetically modified Gardnerella strain comprising the target modification.

16. The method of claim 15, wherein the polynucleotide is a methylated circular polynucleotide.

17. The method of claim 15, wherein the Gardnerella cells are HaeIII-like endonuclease gene-deficient.

18. The method of claim 15, wherein the polynucleotide is a circular polynucleotide containing the target modification and comprising an ermC gene for selection of polynucleotide integration and a mutated pheS gene (T251S A294G) for selection of polynucleotide loss, optionally wherein the mutated pheS gene is codon optimized for Bifidobacterium longum.

19. The method of claim 15, wherein the target modification is an insertion, a point mutation, a deletion, or a combination thereof.

20. The method of claim 15, wherein the polynucleotide is an E. coli plasmid.

21. The method of claim 21, wherein the E. coli plasmid comprises an ermC gene, a polylinker, an E. coli origin of replication, and optionally two copies of a neisserial DNA Uptake Sequence (DUS).

22. The method of claim 15, wherein the polynucleotide is a linear polynucleotide comprising an antibiotic resistance marker.


SEQUENCE LISTING (42 sequences)

SEQ ID NO: 1 - DNA, 31 bp, synthetic construct
  source 1..31: mol_type = other DNA
ctctgagctcctcaaattgtgaacttcaagc

SEQ ID NO: 2 - DNA, 18 bp, synthetic construct
  source 1..18: mol_type = other DNA
tgaacgctcaacacttgc

SEQ ID NO: 3 - DNA, 25 bp, synthetic construct
  source 1..25: mol_type = other DNA
catatgaagagtacaaagttctacc

SEQ ID NO: 4 - DNA, 27 bp, synthetic construct
  source 1..27: mol_type = other DNA
aacagctatgaccatgattacgccaag

SEQ ID NO: 5 - DNA, 21 bp, synthetic construct
  source 1..21: mol_type = other DNA
cgtcaaggataacgaagtagc

SEQ ID NO: 6 - DNA, 21 bp, synthetic construct
  source 1..21: mol_type = other DNA
gctacttcgttatccttgacg

SEQ ID NO: 7 - DNA, 29 bp, synthetic construct
  source 1..29: mol_type = other DNA
gatagagctcggatctaactggcaagaga

SEQ ID NO: 8 - DNA, 28 bp, synthetic construct
  source 1..28: mol_type = other DNA
gctcatcgatcaaaggtctgtcaccatc

SEQ ID NO: 9 - DNA, 21 bp, synthetic construct
  source 1..21: mol_type = other DNA
aggcattggactgcaaactgg

SEQ ID NO: 10 - DNA, 29 bp, synthetic construct
  source 1..29: mol_type = other DNA
ctatgagctcgcatcatggctagaggatc

SEQ ID NO: 11 - DNA, 29 bp, synthetic construct
  source 1..29: mol_type = other DNA
tgatctcgagcacatcggaaaccatgtca

SEQ ID NO: 12 - DNA, 28 bp, synthetic construct
  source 1..28: mol_type = other DNA
gtatctcgagtctgggttgtttgacacg

SEQ ID NO: 13 - DNA, 18 bp, synthetic construct
  source 1..18: mol_type = other DNA
tgtagcgtattgtcgtcg

SEQ ID NO: 14 - DNA, 28 bp, synthetic construct
  source 1..28: mol_type = other DNA
agatatcgattaacgtgactggcaagag

SEQ ID NO: 15 - DNA, 31 bp, synthetic construct
  source 1..31: mol_type = other DNA
gacaatgcatacagctatgaccatgattacg

SEQ ID NO: 16 - DNA, 28 bp, synthetic construct
  source 1..28: mol_type = other DNA
ctatactagtctgtaacttccgcagtga

SEQ ID NO: 17 - DNA, 19 bp, synthetic construct
  source 1..19: mol_type = other DNA
ttcgatgcagtacacaagg

SEQ ID NO: 18 - DNA, 19 bp, synthetic construct
  source 1..19: mol_type = other DNA
tgtgtgatgcgtgaccttc

SEQ ID NO: 19 - DNA, 20 bp, synthetic construct
  source 1..20: mol_type = other DNA
gctggagaagttgtagttcc

SEQ ID NO: 20 - DNA, 18 bp, synthetic construct
  source 1..18: mol_type = other DNA
gaccagaacaggttgagg

SEQ ID NO: 21 - DNA, 29 bp, synthetic construct
  source 1..29: mol_type = other DNA
catagagctcagcacgcagtaaatagacc

SEQ ID NO: 22 - DNA, 33 bp, synthetic construct
  source 1..33: mol_type = other DNA
gcatactagtcgctgttccaatcattatattcc

SEQ ID NO: 23 - DNA, 36 bp, synthetic construct
  source 1..36: mol_type = other DNA
cagtactagtggaatcaacacaactattccttatgc

SEQ ID NO: 24 - DNA, 28 bp, synthetic construct
  source 1..28: mol_type = other DNA
ttaactcgagaactaagatgccctcggt

SEQ ID NO: 25 - DNA, 44 bp, synthetic construct
  source 1..44: mol_type = other DNA
cgtgtgtacaccaccactcctcggaagccaaactctgcgcttcg

SEQ ID NO: 26 - DNA, 44 bp, synthetic construct
  source 1..44: mol_type = other DNA
cgtgtgtacaccaccactccacgcaagccaaactctgcgcttcg

SEQ ID NO: 27 - DNA, 84 bp, synthetic construct
  source 1..84: mol_type = other DNA
cgtggcgtgtgcactcgtgtgtacaccaccactcctcggaagccaaactctgcgcttcgtaaggttgctcgtgtgcgcctcagc

SEQ ID NO: 28 - DNA, 84 bp, synthetic construct
  source 1..84: mol_type = other DNA
cgtggcgtgtgcactcgtgtgtacaccaccactccacgcaagccaaactctgcgcttcgtaaggttgctcgtgtgcgcctcagc

SEQ ID NO: 29 - DNA, 220 bp, synthetic construct
  source 1..220: mol_type = other DNA
ctatgagctcgcatcatggctagaggatcaagattattggagactacacggcattttccgttccgttgaactgtgtgcacatccttccacccatgtatcaaatcttcatgttgatgcagattatagcaatgatacaaatactgggaaactcgcattcagagcaaacattgaaggagacaatttaaaagatattacactacacgcttacatctcgaggtat

SEQ ID NO: 30 - DNA, 1262 bp, synthetic construct
  source 1..1262: mol_type = other DNA
ctgaatcgatcgtaatgcgtcgtacataccgcacattaaaatgaacatgttagtttaaataacgaagaccaaaacgtattgttaaaaatcgcgaaaaatcgctagaactatgcgttaaggtttcactcaagggagatcccatatgcttcacaaaagtacacataacacggataggaagggtgctgtggcaagtagtaagccgttcgacgcgaaggcaattacaaaggtagtaaaagagggtatagcttgtgttcaagccgctaaaacaatggaagagttgaaagcggcaaaaactaagtatgcaggagctcagtctgcaatgacccttgccagcaagtctattggaagtcttcaagtagaagaaaagaaagaagctggcaagataatgtcggctttacgcgctgattttggtcgtgaatttgcagcagcccaagagcgtattaaggctattgaagaagctaatatgcttcaaaaagaaacagtcgatatgaccttgccaataaatcgtaagccgcttggcgctcgccatccgattgcgcgtattattgaagactttgaagacttctttgtgtctatgggttggcagatttctgcaggaccagaggtagaaacagagtggttcgattttgacgcgttgaattttggtccagatcatcctgctcgtcaaatgcaagatactttttatgtgcaaggtaatcaggcaaaggatgctgcaggatttgttggatctaacatggttttgcgtacgcaaacttcttccgatcaggttcgtgcgttaattgagcgaggagtacctctgtatattgcgtccccaggccgagtattccgcacggatgagcttgatgcaacgcatactccagtcttccaccagtgcgaagctttggcagtagataaacatttgagcatggctgatttaaagggtgttcttgatcgccttgctgttgcaatgtttggtccagatgcaaaaagccgtttgcgcccaagctacttcccgttctctgagcctagtgcggagttggatctttggttcccagataagaagggtggcccaggttggattgaatggggcggatgcggaatggtgaatccgaatgttcttaagtctgcaggcttagacccagatgtttatacgggatttggattcggtgttggcttggagcgcactttgctgcttcgtcacgatattaacgatatgcacgatttggttgaaggcgataagcgcttcagtgaacagtttgtgatgggagagtagctcgagtatg

SEQ ID NO: 31 - DNA, 718 bp, synthetic construct
  source 1..718: mol_type = other DNA
ctgtactagtgaaccagctacatcttgcgcagctaagaaagactcgttgaataattatttgtgggatttgcaatacgataaaacaaacattctcgcccgtcatggcgaaaccattgagaacaaattctccagcgacagcttcaacaagaacggtgaattcgttgttgttgagcatcagaagaagaacatcaccaatacaacttcaaatttgtcggttacttccgccaacgatgatcgcgtatacccaggtgctcttttccgtgctgataagaatttgatggacaatatgccaagcctgatttctgcaaaccgcgctccaataacgttgagcgttgatttgccgggattccacggcggtaaagtgctgtaactgttcagcgcccaaccaagagttctgtaacttccgcagtgaacggcttagtttctaagtggaatgcacaatatggagcaagtcatcatgttgcagctcgcatgcagtacgattctgcaagcgcacaaagcatgaaccagctcaaggctaagtttggtgctgattttgccaagattggtgttccgctgaagattgatttcgatgcagtacacaagggtgagaagcagactcaaattgtgaacttcaagcaaacttactacaccgtaagcgttgatgcaccagatagcccagcagatttctttgctccttgcactacgccagacagcttgaagaaccgtatcgattgag

SEQ ID NO: 32 - DNA, 1262 bp, synthetic construct
  source 1..1262: mol_type = other DNA
gttactcgagcgtaatgcgtcgtacataccgcacattaaaatgaacatgttagtttaaataacgaagaccaaaacgtattgttaaaaatcgcgaaaaatcgctagaactatgcgttaaggtttcactcaagggagatcccatatgttgcataaatctacgcacaacactgaccgcaaaggagccgtggcgtctagcaagccgttcgacgctaaagctattactaaagttgtgaaagaaggtatagcctgcgtacaggcagctaaaacaatggaagagttgaaggcggctaagacgaagtatgccggcgctcaatccgctatgacacttgcaagcaaaagcattggatccttgcaagttgaggagaagaaggaagctggtaagattatgtctgctttgcgcgcggattttggccgcgagttcgctgcagcccaagagcgcattaaagcgattgaagaggctaatatgcttcaaaaggaaacggtagatatgacattgccgataaaccgcaaaccgcttggagcacgtcatccgattgcgcgtattattgaagatttcgaggacttcttcgtgtctatgggatggcaaatttctgcgggtccggaagttgagacggagtggtttgatttcgacgcgttgaatttcggtccagaccacccagctcgtcagatgcaggataccttctacgtacaaggaaatcaggccaaagatgcggctggatttgttggatcgaatatggtgcttcgtacacagacgtcttctgatcaagttcgtgcattgattgaacgtggtgttccactttatattgcgagcccgggcagggtgttcaggactgacgaacttgacgcgactcacaccccagtgtttcatcaatgcgaggcgcttgctgtggataagcatctttccatggccgaccttaaaggcgttcttgataggcttgctgttgcaatgttcggcccagacgcgaagtctcgtctgcgcccgtcttactttccgtttagcgaaccgtctgcggaattggacctttggtttccggacaaaaaaggaggaccaggctggatagaatggggtggatgcggcatggtgaacccaaatgtacttaaatccgctggtcttgacccagacgtttacacgggatttggttttggtgtcggtcttgaacgcacactgttgctgaggcatgacattaacgatatgcatgacttggtagagggcgacaaacgtttctctgagcagtttgttatgggtgagtagggtacctcag

SEQ ID NO: 33 - DNA, 995 bp, synthetic construct
  source 1..995: mol_type = other DNA
gtcgactagtccagactcacttctaggagcgccagaaagcgcttcttcttgagtcaatggcgtagcaggctcgtctgggcgcaaatacgcttcaagaggtactggatttgcagtaggtggcacatcttcgccagcaacttcctgcactttgttaatggattctgcaatgacgtttaattcgccttgcaagcgactgatttcttcatcgcttaaagcaatctgggacaaaacacccaaatgctcaatttcttcgcgtgtgaatgtaggcataacctcaactatatgtgtgatgcgtgaccttcttatagaagaaaaaagcaatattgataaaagatttacacacgttactgtagaaatattttcccaattttagaacatgattgtcgatccatattttctagggaaaattatgtgtaatattaaaaaaaattttattagtgagacaataagcgaaataacaattattgtcacatacagttttcataaaattaacgttatttgcagaatatttaaatattttagatatcgcgctaaatatcgcgatgcttttccacaacgtggccaattgcatacatgactacgccccaagcaagaaccaccaagcctgattgccaccatgtaaaaacatatgcgtttggtggaagctgtgcacccgaattaggggagccgcccaagaatttctccaccgctgtggctggcaaaagctgtataagtattgaattccacttcgcgaaattgcttgcaaacataataatgctaagaacactaggcaaaatcatcacggctccaataacgcacataattccgccagcagttgacttgcaaatcatgccaaagccgtacgccattgctgctacaacaaccataattgcaggagagccaagaaacagcgtaagaggcaatttccacgcgttgcttccagataatccagaagtgttgtctcctataaaggctaattctgcagcgcctagcgaaactgccattgcccgggtata

SEQ ID NO: 34 - DNA, 2088 bp, synthetic construct
  source 1..2088: mol_type = other DNA
atatgagctcgctcttggtcttatcccagatattttggaagatgataaatcttctcaaccagaaggaacttttacaaaacagcttcctaaaggtggagctaaagtttcttcaggatctagagtaagcgtatggttctctgtaggtccacagtctactaagattccagatgttactggtaaatcgcaagatgtagcacgtaaagctcttgaacgtgctggatttaagatatctaatgttcgtgtagaagatagcacggaagttaagaagaatcatgttactcgtacagatccttctgcagattcgtttgcagacaaggggtctatggttaccttatacatttcttctggtcttacaaagatccctgatggtttagttggacaatctaaggatgttgtaacaagcgaattgcaaaatcttggttttactgtaaatgttgttgaggaagaatctgatactgcatctgaaggaactgtgaccaagatgaacccatcttctggtgctgcagtaaagcctcatagctctgtaacagtatatgtttctaaaggaaagcctaaagttgaagttcctcgtttggctgttggcactgtaactttcaagcaggctaaacaagtattggaagcaaaaggctttaaggttgttgcatctgatgcatcagctaaagatgatgacatagttactggaatgtctgaaaaagaaggagcaaagattgacaaaggttcaacaatcactttgactgtgaaatctgctactcctccatcggaccttacaaagccaggtacgggattagatttgaataaaccttctactgaagccccatctacatctactacaacaacagatacaggtttgtaaaaagcaaagcataaagccagtaagtttaataagcttactggctttccatggttagtattaagcgtctgatatgattatcttttgcgtgtccattgagcatgaatgttgtcgcgttatatgattgctcgttttttattgtctactcggatacgcgaatacgcgccatgtacgcgtccgttcgtgtcggtcggcttggatttagacatatgttatccaagttgcatgcgaacaggcatggccaggcaagaaccgacaccggtctccaccgcggtggcggccgcactagtggatcccccgggctgcaggaattctgtacacctaggatatcaagcttatcgataccgtcgacggccggcctttattatttattaatcttatctacttacttattatctacttaccccttagtaaggcgttgctgtgtatgcagacatttctcttaagaatggtatatttgccgaaacaaacgggtatacccattgcataagtatcataatgcatattaggtagataattaaggagagaattaatctcactggaagaattccaggctgtagcctcatgagtccgccaaagatgctgaattcaggctttggctttagacctgctttaatctcgcgtcttaatggccactgccatgcaatagctccagcagcaaagataatcaaataaactatcagcgcgccaatcataagcggaacaagcgaatcgagatgagagatcaaagattgttgctcgttattaacaaacttaacttttccgttttcatctaaagtagaaagctcttttggaataccgtcagaaactttagcccaatagtcaagctctccaaagctaacaaagcggaacttaggtgtagagaacttaggttcgcatgtaataatggtgatcatgcgtttctttggctctttactatggtttattggatctgggtcaagaacctcaacctgttctggtctaacaatcttgtgagtaatgtatttgtaaacgaaccaataatctttagttcgaagaataatagaatctcccttttggaacctatcaacatccgcaagaggctgaccataaccattgcggtgaccaataattgtaatgtttccgatttctccaggcaactcagttttaggataatgacctaatcctcgacgattaagtatctccatatctacaccttcaataacattacgctcccattgatcaccgaagcgtggaatatagattcgtgctcgagtatc

SEQ ID NO: 35 - DNA, 51 bp, synthetic construct
  source 1..51: mol_type = other DNA
gatttgccggggattccacggcggctaaagtgctgtaactgttcagcgccc

SEQ ID NO: 36 - protein, 16 aa, synthetic construct
DLPGFHGGSAVTVQRP

SEQ ID NO: 37 - DNA, 978 bp, Gardnerella vaginalis
  source 1..978: mol_type = genomic DNA
cattggactgcaaactggaagatatcatggaaaacgtagaagaataaggagattttaaatgattattagaacatatggttggattattgaaaatctgtatcgctttaaactgcaatattagtgacattatggaaatacaaaaagaaatgaggtaaaaaaaattatggcaaaaacatttggttccgaagaacataaaatattatcactgtttagcgctgactcaacattttgttatgaaggagaattctttaaagtaaataattctggaaagccaacctgcaaaaaaggtgaacctaaaactgatatatatgtagaagctgttaatagtaacaactgtgttaaagagtttaaaatctcttttaagcaagaaaatgctgaatttcttgaaaataaaaccaatgctgagcgagcagaacagttattcggatctaactggcaagagattattatcagcgctgtgactaaattacagaatgattttcaaaaaagacccattgatgattacgttgatcagaatccaaatgtgtattttgcttgtaaagcgttaaattatcgttcacttaaaaataaatatgatggtgacagacctttggctgtttatgtaaattggttcgtaactaatggaaaattggattatgaattagtatttgataaaccccttttggaagggggagatgctgtatatgaacgattaaaatcagctttagatttattaggcatttccaatactaatgatttaagtcttgaattaataatgcatactgaatctgtgaacatataggcatagatgagtggtggaagctttaaactcagctcccaccacttatctgaattatgatagaacttttttaattgcagtagctatttcaaatgctagatttactggtactgcattcccaatcattttataacccgtatctaagctatcgtatatgaacttaaaattatctggaaatccttgtattctggctatttctc

SEQ ID NO: 38 - DNA, 4249 bp, synthetic construct
  source 1..4249: mol_type = other DNA
gcggccgtaaaacgacggccagtgagcgcgcgtaatacgactcactatagggcgaatttctagaggagctccaccgcggtggcggccgcactagtcattggactgcaaactggaagatatcatggaaaacgtagaagaataaggagattttaaatgattattagaacatatggttggattattgaaaatctgtatcgctttaaactgcaatattagtgacattatggaaatacaaaaagaaatgaggtaaaaaaaattatggcaaaaacatttggttccgaagaacataaaatattatcactgtttagcgctgactcaacattttgttatgaaggagaattctttaaagtaaataattctggaaagccaacctgcaaaaaaggtgaacctaaaactgatatatatgtagaagctgttaatagtaacaactgtgttaaagagtttaaaatctcttttaagcaagaaaatgctgaatttcttgaaaataaaaccaatgctgagcgagcagaacagttattcggatctaactggcaagagattattatcagcgctgtgactaaattacagaatgattttcaaaaaagacccattgatgattacgttgatcagaatccaaatgtgtattttgcttgtaaagcgttaaattatcgttcacttaaaaataaatatgatggtgacagacctttggctgtttatgtaaattggttcgtaactaatggaaaattggattatgaattagtatttgataaaccccttttggaagggggagatgctgtatatgaacgattaaaatcagctttagatttattaggcatttccaatactaatgatttaagtcttgaattaataatgcatactgaatctgtgaacatataggcatagatgagtggtggaagctttaaactcagctcccaccacttatctgaattatgatagaacttttttaattgcagtagctatttcaaatgctagatttactggtactgcattcccaatcattttataacccgtatctaagctatcgtatatgaacttaaaattatctggaaatccttgtattctggctatttctcccatcaagcttatcgataccgtcgacctcgagcgtaatgcgtcgtacataccgcacattaaaatgaacatgttagtttaaataacgaagaccaaaacgtattgttaaaaatcgcgaaaaatcgctagaactatgcgttaaggtttcactcaagggagatcccatatgttgcataaatctacgcacaacactgaccgcaaaggagccgtggcgtctagcaagccgttcgacgctaaagctattactaaagttgtgaaagaaggtatagcctgcgtacaggcagctaaaacaatggaagagttgaaggcggctaagacgaagtatgccggcgctcaatccgctatgacacttgcaagcaaaagcattggatccttgcaagttgaggagaagaaggaagctggtaagattatgtctgctttgcgcgcggattttggccgcgagttcgctgcagcccaagagcgcattaaagcgattgaagaggctaatatgcttcaaaaggaaacggtagatatgacattgccgataaaccgcaaaccgcttggagcacgtcatccgattgcgcgtattattgaagatttcgaggacttcttcgtgtctatgggatggcaaatttctgcgggtccggaagttgagacggagtggtttgatttcgacgcgttgaatttcggtccagaccacccagctcgtcagatgcaggataccttctacgtacaaggaaatcaggccaaagatgcggctggatttgttggatcgaatatggtgcttcgtacacagacgtcttctgatcaagttcgtgcattgattgaacgtggtgttccactttatattgcgagcccgggcagggtgttcaggactgacgaacttgacgcgactcacaccccagtgtttcatcaatgcgaggcgcttgctgtggataagcatctttccatggccgaccttaaaggcgttcttgataggcttgctgttgcaatgttcggcccagacgcgaagtctcgtctgcgcccgtcttactttccgtttagcgaaccgtctgcggaattggacctttggtttccggacaaaaaaggaggaccaggctggatagaatggggtggatgcggcatggtgaacccaaatgtacttaaatccgctggtcttgacccagacgtttacacgggatttggttttggtgtcggtcttgaacgcacactgttgctgaggcatgacattaacgatatgcatgacttggtagagggcgacaaacgtttctctgagcagtttgttatgggtgagtagggtaccggggcagcttttgttccctttagtgagggttaattgcgcgcttggcgtaatcatggtcatagctgttcgataagctaattctcatgtttgacagcttatcatcgcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgactctagagcttgccgtctgaaatggtttcagacggcatgcagcaatggcaacaacgttgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggcggttttttgtttgcaagcagcagattacgcgcagaaaaaaaggatctcaagaagatcctttgatcttttcttactgaacggtgatccccaccggaatt

SEQ ID NO: 39 - DNA, 669 bp, synthetic construct
  source 1..669: mol_type = other DNA
gaataagactgagggtgaggattctaagaagactgcgaccgagactaataagaaggaagaatctaagaaggagcagcctgagcagactaaaccaactgagcagactgtagtaaaaaccgaaggtaaggctgacgctactaagactgagtctgggtcgaaggttgagagcgctacggctgtagataataaggttacgaatagcacttctgctactgataagaataatgcagacagtagcaccactgaggaaactagcactcctgcggaaaatgttgtaaagaagcgtacaaggcgtgcgacggttgagaataagcaacctgaggctactaattatgctaaggatgaaaatcagaagcagagcgaacctgttgttggtcaagatcgcgaagctaatcctgcacctgctccacaagttaatagtgagataacgctttaaaaagaaacaaaagacacgcttcctaacttgtatgcatggggatcttctgacaatgtgtatattgaaaaagggcaaaaccaagaagtaacatttaattttgcaaaaccttctgacggtagcacaattaccaaagtagcaattttcccatctgatggtaatactgttgataatgataaaagtagaaagttccttgaatattattctgctaatgaaaacgagcacaagccatattc

SEQ ID NO: 40 - DNA, 3781 bp, synthetic construct
  source 1..3781: mol_type = other DNA
gcggccgtaaaacgacggccagtgagcgcgcgtaatacgactcactatagggcgaatttctagaggagctccaccgcggtggcggccgcactagttaataaggttacgaatagcacttctgctactgataagaataatgcagacagtagcaccactgaggaaactagcactcctgcggaaaatgttgtaaagaagcgtacaaggcgtgcgacggttgagaataagcaacctgaggctactaattatgctaaggatgaaaatcagaagcagagcgaacctgttgttggtcaagatcgcgaagctaatcctgcacctgctccacaagttaatagtgagataacgctttcaaaagaaacaaaagacacgcttcctaacttgtatgcatggggatcttctgacaatgtgtatattgaaaaagggcaaaaccaagaagtaacatttaattttgcaaaaccttctgacggtagcacaattaccaaagtagcaattttcccatctgatggtaatactgttgataatgataaaagtagaaagttccttgaatattattctgctaatgaaaacgagcacaagccatattctggtaagtacgaatttgaattcgatatcaagcttatcgataccgtcgacctcgagcgtaatgcgtcgtacataccgcacattaaaatgaacatgttagtttaaataacgaagaccaaaacgtattgttaaaaatcgcgaaaaatcgctagaactatgcgttaaggtttcactcaagggagatcccatatgttgcataaatctacgcacaacactgaccgcaaaggagccgtggcgtctagcaagccgttcgacgctaaagctattactaaagttgtgaaagaaggtatagcctgcgtacaggcagctaaaacaatggaagagttgaaggcggctaagacgaagtatgccggcgctcaatccgctatgacacttgcaagcaaaagcattggatccttgcaagttgaggagaagaaggaagctggtaagattatgtctgctttgcgcgcggattttggccgcgagttcgctgcagcccaagagcgcattaaagcgattgaagaggctaatatgcttcaaaaggaaacggtagatatgacattgccgataaaccgcaaaccgcttggagcacgtcatccgattgcgcgtattattgaagatttcgaggacttcttcgtgtctatgggatggcaaatttctgcgggtccggaagttgagacggagtggtttgatttcgacgcgttgaatttcggtccagaccacccagctcgtcagatgcaggataccttctacgtacaaggaaatcaggccaaagatgcggctggatttgttggatcgaatatggtgcttcgtacacagacgtcttctgatcaagttcgtgcattgattgaacgtggtgttccactttatattgcgagcccgggcagggtgttcaggactgacgaacttgacgcgactcacaccccagtgtttcatcaatgcgaggcgcttgctgtggataagcatctttccatggccgaccttaaaggcgttcttgataggcttgctgttgcaatgttcggcccagacgcgaagtctcgtctgcgcccgtcttactttccgtttagcgaaccgtctgcggaattggacctttggtttccggacaaaaaaggaggaccaggctggatagaatggggtggatgcggcatggtgaacccaaatgtacttaaatccgctggtcttgacccagacgtttacacgggatttggttttggtgtcggtcttgaacgcacactgttgctgaggcatgacattaacgatatgcatgacttggtagagggcgacaaacgtttctctgagcagtttgttatgggtgagtagggtacctggggcagcttttgttccctttagtgagggttaattgcgcgcttggcgtaatcatggtcatagctgttcgataagctaattctcatgtttgacagcttatcatcgcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgactctagagcttgccgtctgaaatggtttcagacggcatgcagcaatggcaacaacgttgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggcggttttttgtttgcaagcagcagattacgcgcagaaaaaaaggatctcaagaagatcctttgatcttttcttactgaacggtgatccccaccggaatt

SEQ ID NO: 41 - DNA, 1077 bp, synthetic construct
  source 1..1077: mol_type = other DNA
gaagattgcgtgaactggaacatgctgctgagcgtagtgtgcgtgattggcacgggcaccatggcggctgggttcttccttgatcagaccaagtgggggcgcgcgatgcttgatgcgttgttgcgtaagacactgattaaagattttatctgcaaaactgccaaaaagcttcgcgctttaaggcgacgtagcgaaaggtggcgatgctgatgaaaaaatttgcacgcaatcctgctagtcggctagtcgaaagagtagcgctggcttgcaagcttgcggtgcgattctcgctaaaacttgttgcgtactacgattcgcgtaaacattagcgcgatgaataattgtggctcgttcgcaaattgtggattgatcacgaaccgtggcttgcttacagattgtggcttgcctatatatcgcaacacaaaataaacaataaacattaaacaataaccgtattttcctcaaaaacctgcccaaaagggcactgatataaaggagaagaaagtgactagttgatttgctcagtttgattagtaaatcataaacgattgagtctagggcaaaaaccctagactcaatcctatgtggactgttaaagcaacaaaacccgtcgttttccgctttttgttgttgattttgtggttttaagcaacattttcggtaagtttttcccgattttgttgttaattttgccgttttgagcaacaaaaagcgcaaaatcccactgcgtttgtagcaaaatcaaaccaaatattatctttaattcaatagcttgagcttggaggagttatgaagctagccgatattagcaatttgtgccactcgttgagtgagcgcgctgttgctaaattgtttaagaataaggcgaagcgtacctccaatgtggctggcacgagcagtgtggctggtgcgagtggtttgctagcggataagtcgcaagcaaccgaagagcaaattccacgatccaactcaagcaaaaatataaaccaaaatacacttaaaactaaaaaaataactaaaaagaaaagcacactgatgcgtatcttagagccgattgcgctggttcttgcaggcatt

SEQ ID NO: 42 - DNA, 4287 bp, synthetic construct
  source 1..4287: mol_type = other DNA
gcttcaaaaggaaacggtagatatgacattgccgataaaccgcaaaccgcttggagcacgtcatccgattgcgcgtattattgaagatttcgaggacttcttcgtgtctatgggatggcaaatttctgcgggtccggaagttgagacggagtggtttgatttcgacgcgttgaatttcggtccagaccacccagctcgtcagatgcaggataccttctacgtacaaggaaatcaggccaaagatgcggctggatttgttggatcgaatatggtgcttcgtacacagacgtcttctgatcaagttcgtgcattgattgaacgtggtgttccactttatattgcgagcccgggcagggtgttcaggactgacgaacttgacgcgactcacaccccagtgtttcatcaatgcgaggcgcttgctgtggataagcatctttccatggccgaccttaaaggcgttcttgataggcttgctgttgcaatgttcggcccagacgcgaagtctcgtctgcgcccgtcttactttccgtttagcgaaccgtctgcggaattggacctttggtttccggacaaaaaaggaggaccaggctggatagaatggggtggatgcggcatggtgaacccaaatgtacttaaatccgctggtcttgacccagacgtttacacgggatttggttttggtgtcggtcttgaacgcacactgttgctgaggcatgacattaacgatatgcatgacttggtagagggcgacaaacgtttctctgagcagtttgttatgggtgagtagggtacccagcttttgttccctttagtgagggttaattgcgcgcttggcgtaatcatggtcatagctgttcgataagctaattctcatgtttgacagcttatcatcgcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgactctagagcttgccgtctgaaatggtttcagacggcatgcagcaatggcaacaacgttgaagctagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgtcgctggcgtttttccataggctccgcccccctgacgagcatcacaaaaatcgacgctcaagtcagaggtggcgaaacctgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccgtaaacgacggccagtgagcgcgcgtaatacgactcactatagggcgaattggagctcgaagattgcgtgaactggaacatgctgctgagcgtagtgtgcgtgattggcacgggcaccatggcggctgggttcttccttgatcagaccaagtgggggcgcgcgatgcttgatgcgttgttgcgtaagacactgattaaagattttatctgcaaaactgccaaaaagcttcgcgctttaaggcgacgtagcgaaaggtggcgatgctgatgaaaaaatttgcacgcaatcctgctagtcggctagtcgaaagagtagcgctggcttgcaagcttgcggtgcgattctcgctaaaacttgttgcgtactacgattcgcgtaaacattagcgcgatgaataattgtggctcgttcgcaaattgtggattgatcacgaaccgtggcttgcttacagattgtggcttgcctatatatcgcaacacaaaataaacaataaacattaaacaataaccgtattttcctcaaaaacctgcccaaaagggcactgatataaaggagaagaaagtgactagttgatttgctcagtttgattagtaaatcataaacgattgagtctagggcaaaaaccctagactcaatcctatgtggactgttaaagcaacaaaacccgtcgttttccgctttttgttgttgattttgtggttttaagcaacattttcggtaagtttttcccgattttgttgttaattttgccgttttgagcaacaaaaagcgcaaaatcccactgcgtttgtagcaaaatcaaaccaaatattatctttaattcaatagcttgagcttggaggagttatgaagctagccgatattagcaatttgtgccactcgttgagtgagcgcgctgttgctaaattgtttaagaataaggcgaagcgtacctccaatgtggctggcacgagcagtgtggctggtgcgagtggtttgctagcggataagtcgcaagcaaccgaagagcaaattccacgatccaactcaagcaaaaatataaaccaaaatacacttaaaactaaaaaaataactaaaaagaaaagcacactgatgcgtatcttagagccgattgcgctggttcttgcaggcattctcgagcgtaatgcgtcgtacataccgcacattaaaatgaacatgttagtttaaataacgaagaccaaaacgtattgttaaaaatcgcgaaaaatcgctagaactatgcgttaaggtttcactcaagggagatcccatatgttgcataaatctacgcacaacactgaccgcaaaggagccgtggcgtctagcaagccgttcgacgctaaagctattactaaagttgtgaaagaaggtatagcctgcgtacaggcagctaaaacaatggaagagttgaaggcggctaagacgaagtatgccggcgctcaatccgctatgacacttgcaagcaaaagcattggatccttgcaagttgaggagaagaaggaagctggtaagattatgtctgctttgcgcgcggattttggccgcgagttcgctgcagcccaagagcgcattaaagcgattgaagaggctaatat